(19) United States
(12) Patent Application Publication
(10) Pub. No.: US 2003/0216758 A1
(43) Pub. Date: Nov. 20, 2003

(54) Coated surgical patches

(75) Inventor: Pierre E. Signore, Vancouver (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Angiotech Pharmaceuticals, Inc., Vancouver (CA)

(21) Appl. No.: 10/331,137

(22) Filed: Dec. 27, 2002

Related U.S. Application Data
(60) Provisional application No. 60/344,011, filed on Dec. 28, 2001.

(51) Int. Cl. A61B 17/08

(52) U.S. Cl. 606/151

(57) ABSTRACT

Surgical patches are described which release an anti-inflammatory agent, an anti-platelet agent, an anticoagulant, a fibrinolytic agent, a cell-cycle inhibitor, and/or an anti-proliferative agent.

### CROSS-REFERENCE TO RELATED APPLICATION

This application claims the benefit of U.S. Provisional Patent Application No. 60/344,011, filed Dec. 28, 2001, where this provisional application is incorporated herein by reference in its entirety.

### BACKGROUND OF THE INVENTION

1. Field of the Invention

This invention relates to surgical patches coated with biologically active agents to prevent adverse tissue reaction to the patch.

2. Description of the Related Art

Primary closure and patch angioplasty are two techniques of arteriotomy closure used by surgeons after vascular procedures. In primary closure, the lips of the arterial wound are directly sutured to each other whereas an extra piece of material is sutured between the two lips during patch angioplasty. Patch angioplasty is preferred after procedures with a high rate of postoperative narrowing of the repaired vessel (endarterectomy of small carotid arteries for example). The added piece of material maintains the original diameter of the blood vessel and induces favorable local hemodynamics that otherwise may lead to recurrent stenosis.

Patch angioplasty can be performed with autologous tissue (typically the patient's saphenous vein) or synthetic material (expanded polytetrafluoroethylene or Dacron). Vein patches have drawbacks such as aneurismal degeneration and rupture. They require an additional incision to harvest the vein with associated morbidity. The patient veins may not be suitable for patching. Most importantly, the vein used for the patch will not be available for coronary artery bypass grafting should the patient require arterial reconstruction at a later time. For these reasons, the use of synthetic patches has become increasingly popular.

However, synthetic materials implanted in the vasculature induce thrombogenic, inflammatory and hyperproliferative responses. Immediately after implantation, platelets bind to the luminal surface of the prosthesis, triggering the coagulation cascade and inducing thrombus formation. Thrombus may grow large enough to cause distal ischemia (stroke in the case of carotid artery patches).

In the days following the procedure, inflammatory cells such as macrophages, lymphocytes and neutrophils adhere to the prosthetic lumen and also migrate into the peri-prosthetic space. These cells release cytokines that promote smooth muscle cell migration from the adjacent vessel on the luminal surface of the patch. The cells further proliferate on the patch and secrete extracellular matrix. Depending on the porosity of the patch material, cells may also migrate through the pores of the patch from the surrounding tissue into the lumen. In both cases, hyperplasia causes plaque formation on the luminal surface of the patch and the adjacent vessels within a few weeks. This reduces luminal area in the treated blood vessel thus impeding blood flow to the distal tissues.

Therefore, there exists a need for a means and a method to prevent inflammatory reaction, thrombus formation and intimal hyperplasia on the luminal surface of synthetic patches. The present invention meets this need, and further, provides other, related advantages.

### SUMMARY OF THE INVENTION

Briefly stated, the present invention involves methods of making and using surgical patches which release agents that prevent inflammatory reactions, thrombus formations and/or intimal hyperplasia. Representative examples of such agents include cell-cycle inhibitors such as taxanes, camptothecins, doxorubicin, immunosuppressive drugs (rapamycin, cyclosporines), bromocryptine, tubercidine, beta-lapachone, glucocorticoids, nonsteroidal anti-inflammatory drugs, cell cycle inhibitors, calcium channel blockers, calcium chelating agents, inhibitors of matrix metalloproteinases, methotrexate, thrombolytic agents, anti-platelet agents and anticoagulation agents. The presence of these agents, alone or in combination, on the patch will effectively prevent or inhibit local inflammatory reaction, prevent thrombus material from building up on the patch and stop cells from proliferating onto the patch.

Thus, within one aspect of the present invention surgical patches (e.g., vascular patches) are provided which release an anti-inflammatory agent, an anti-platelet agent, an anticoagulant agent, fibrinolytic agents, a cell-cycle inhibitor agent, and/or an anti-proliferative agent. Within certain embodiments, the vascular patch is a synthetic patch (e.g., made of Dacron). Within various embodiments, the anti-inflammatory agent is aspirin, ibuprofen, or a glucocorticoid drug, the anti-coagulant agent is heparin or hirudin, and the fibrinolytic agent is tissue plasminogen activator, streptokinase, or urokinase. Within other embodiments, the cell-cycle inhibitor agent is a taxane (e.g., paclitaxel or docetaxel), a vinca alkaloid (e.g., vinblastine or vincristine), a podophyllotoxin (e.g., etoposide), an anthracycline (e.g., doxorubicin or mitoxantrone), or a platinum compound (e.g., cisplatin or carboplatin).

Also provided are methods for making surgical patches (e.g., vascular patches) which release an anti-inflammatory agent, an anti-platelet agent, an anticoagulant, an anti-fibrinolytic agent, a cell-cycle inhibitor, and/or an anti-proliferative agent, comprising the step of coating at least a part (all or a portion such as the ends, or one side) of the patch (e.g., by spraying or dipping) with one of the factors or agents mentioned above. Alternative methods for generating patches (e.g., interweaving a patch with a coated thread, or absorbing a desired agent onto the patch) are described in more detail below. Within further embodiments, the factor or agent may be mixed or formulated with another compound or carrier (e.g., polymeric or non-polymeric). In one embodiment of the present invention, only one side of the patch is coated leaving the other side and most of the thickness of the patch untreated. In another embodiment, only parts (the edge for example) of the patch are coated.

Within other aspects of the invention, methods are provided for closing an opening in the biological tissue (e.g., the vasculature), comprising applying to the opening in a surgical patch as described herein. Within certain embodiments, the compound or composition may be applied by itself or in a carrier, which may be either polymeric, or non-polymeric. Within certain embodiments, the surgical patch is a vascular patch, which is sutured in place.

These and other aspects of the present invention will become evident upon reference to the following detailed description and attached drawings. In addition, various references are set forth herein which describe in more detail certain procedures or compositions (e.g., compounds, proteins, vectors, and their generation, etc.), and are therefore incorporated by reference in their entirety. When PCT applications are referred to it is also understood that the corresponding or cited U.S. applications or U.S. Patents are also incorporated by reference herein.

### BRIEF DESCRIPTION OF THE DRAWINGS

FIG. 1 is a schematic illustration showing sites of action within a biological pathway where Cell Cycle Inhibitors may act to inhibit the cell cycle.

### DETAILED DESCRIPTION OF THE INVENTION

Prior to setting forth the invention, it may be helpful to an understanding thereof to set forth definitions of certain terms that will be used hereinafter.

“Cell Cycle Inhibitor” as used herein refers to any protein, peptide, chemical or other molecule which delays or impairs a dividing cell's ability to progress through the cell cycle and replicate. Cell cycle inhibitors, which prolong or arrest mitosis (M-phase) or DNA synthesis (S-phase), are particularly effective for the purposes of this invention as they increase the dividing cell's sensitivity to the effects of radiation. A wide variety of methods may be utilized to determine the ability of a compound to inhibit the cell cycle including univariate analysis of cellular DNA content and multiparameter analysis (see the Examples).

### I. Patches

Patches are small pieces of material used to mend a tear or a break to cover a hole or to strengthen a weak place. In medicine, surgical patches are pieces of synthetic material or biological tissue used to bridge together the defect between the edge of an incision or a gap in a biological structure (e.g., a vessel wall). Patches are also used after lung surgery to strengthen the repaired lung.

Synthetic vascular patches are available from medical device companies such as IMPRA, WL Gore, Sulzer Vascutek, Shelhigh, Bio Nova International, Intervascular and Aesculap for example. Tissue-based vascular patches are available from Biovascular and St Jude Medical. Representative examples of surgical patches are described in U.S. Pat. Nos. 5,100,422; 5,104,400; 5,437,900; 5,456,711; 5,641,566; 5,645,915; 6,296,657; and 6,322,593.

Vascular patches as described herein can be, among other uses, during vascular surgery to repair blood vessels.

### II. Agents

Anti-Inflammatory Agents

Inflammation occurs when cells of the immune system are activated in response to foreign agents or antigens. Leucocytes release lysosomal enzymes. Arachidonic acid is synthesized and eicosanoids, kinins, complement components and histamine are released. Cytokines have a powerful chemotactic effect on eosinophils, neutrophils and macrophages. They also promote local hyperemia and vascular permeability. Superoxide anion is formed by the reduction of molecular oxygen, which stimulates the production of other reactive molecules such as hydrogen peroxide and hydroxyl radicals. The interaction of these substances with arachidonic acid results in the generation of more chemotatic substances, thus perpetuating the inflammatory process. Anti-inflammatory drugs inhibit one or several of the processes described above thus interfering with the inflammatory reaction.

Examples of anti-inflammatory drugs include but are not limited to nonsteroidal inflammatory drugs such as aspirin, ibuprofen, naproxen, fenoprofen, indomethacin, sulindac, meclofenamate, mefenamic acid, tolmetin, phenylbutazone, piroxicam, diflunisal apazone carprofen, flurbiprofen, diclofenac, ketoprofen; slow-acting anti-inflammatory drugs such as chloroquinine, hydroxychloroquinine, gold, penicillamine, levamisole; glucocorticoid drugs such as hydrocortisone, cortisone, dexamethasone, prednisone, fluocortolone, triamcinolone, fludrocortisone; statins such as pravastatin, fluvastatin, simvastatin, lovastatin; thromboxane inhibitors such as triazolopyrimidine; immunosuppressive agents such as rapamycin, sirolimus, tacrolimus, everolimus, cyclosporin A; anti-inflammatory cytokines such as interleukin-10.

The anti-inflammatory potential of agents can be assessed by studying their inhibition of cyclooxygenase-1 and cyclooxygenase-2 (Everts et al., 2000. Clin. Rheumatol. 19: 331-343), their inhibition of phospholipase activity and prostaglandine release (Sampey et al., Mediators Inflamm. 9:125-132, 2000), their inhibition of tumor necrosis factor-alpha (TNF-α) synthesis and secretion (Joyce et al., Inflamm Res. 46:447-451, 1997), their inhibition of vasodilation and permeability of the microcirculation (Perratti and Ahluwalia, 2000 Microcirculation 7: 147-161), their inhibition of toluene di-isocyanate-induced mast cell proliferation and degranulation, of anti-CD3-induced T-lymphocyte proliferation, of TNF-α-induced cell adhesion molecule expression, of oedema formation, of interleukin-5 (IL-5)-induced blood eosinophilia, of IL-5- or platelet activating factor-stimulated pulmonary eosinophilia, (Johnson, 1995 Allergy 50: 11-14), with neutrophil activation assays (Jackson et al., 1997 Immunology 90: 502-510), with cytokine gene expression assays (White et al., 1998 Cancer Immunol. Immunother. 46: 104-112).

Anti-Platelet Agents

Hemostasis is the spontaneous arrest of bleeding from a damaged blood vessel. The normal vascular endothelium is not thrombogenic and circulating blood platelets and clotting factors do not adhere to it. However, within seconds of damage to a blood vessel, platelets adhere to the site of injury. As platelets become activated, they secrete agents such as ADP and prostaglandins that enhance recruitment and adherence of other platelets. The resulting growing thrombus of aggregated platelets reduces blood flow and triggers fibrin formation. The fibrin network reinforces the initial platelet plug thus ensuring long-term hemostasis. At a later stage, platelets release growth factors such as platelet-derived growth factor that promote healing of the damage blood vessel.

Anti-platelet agents are compounds that interfere with platelet activation, adhesion or secretion and thus inhibit thrombus formation. Examples of anti-platelet agents include but are not limited to, aspirin (Awtry, 2000, Circulation, 101: 1206-1218), ADP receptor antagonists such as clopidogrel, ticlopidine and their active metabolites (Coukell and Markham, 1997 Drugs 54: 745-750; Muller et al., 2000 Circulation 101: 590-593; Bertrand et al., 2000 Circulation 102: 624-629; Quinn and Fitzgerald, 1999 Circulation 100: 1667-1672), serotonin receptor antagonists (Herbert et al., 1993 Thromb. Haemostas. 69: 262-2670), platelet glycoprotein receptor antagonists such as abciximab, tirofiban, eptifibatide, lamifiban, orbofiban, roxifiban, sibrafiban, lefradafiban, xemilofiban and their active metabolites (Dobesh and Latham, 1998 Pharmacotherapy 18: 663-685; Madan et al., 1998 Circulation 98: 2629-2635), statins such as pravastatin, fluvastatin, simvastatin, lovastatin (Igarashi et al., 1997 British Journal of Pharmacology 120: 1172-1178), cAMP phosphodiesterase inhibitors such as cilostazol (Kimura et al., 1985 Drug Res. 35: 1144-1149); nitric oxyde donors such as molsidomine, linsidomine, L-arginine ( ), alpha-adrenergic antagonists such as dihydrogeneted ergopeptines, phentolamine, and yohimbin.

The antiplatelet activity of agents can be assayed by monitoring in vitro platelet aggregation after activation by agonists using turbidimetry or radiolabeled platelets. In vivo quantification of platelet aggregation can be performed with radiolabeled platelets in models of arterio-venous shunts, stent placement and graft implantation. In vivo antiplatelet activity can also be assessed by monitoring arterial temperature distal to thrombus formation and by determining bleeding time. (Hebert et al., 1998 Thromb. Haemost. 80: 512-518; Hebert et al., 1993 Arteriosclerosis and Thrombosis 13: 1171-1179; Harker et al., 1998 Circulation 98: 2461-2469; Yao et al., 1993 Trans. Associa. AU Physicians 106: 110-119).

Anticoagulants

Blood coagulates by the transformation of soluble fibrinogen into insoluble fibrin. More than a dozen circulating proteins interact in a cascading series of proteolytic reactions. At each step, an inactive clotting factor undergoes proteolytic cleavage and become an active protease. This protease activates the next clotting factor. The end product of the coagulation cascade is the formation of a solid fibrin clot.

Anticoagulants are agents that interfere with the coagulation cascade and inhibit the formation of fibrin. Examples of anticoagulants include, but are not limited to, warfarin and coumarin anticoagulants, tissue factor pathway inhibitor, active-site inactivated factor VIIa (DEGR-VIIa), tick anticoagulant peptide, antithrombin agents such as heparin, low-molecular-weight-heparin, hirudin, bivalirudin (Jang et al., 1995 Circulation 92: 3041-3050), retinoids such as all-trans-retinoic acid.

The anticoagulation activity of agents can be assayed by measuring the activated partial thromboplastin time and the prothrombin time (Freund et al., 1993 Thrombosis and Hemostasis 69: 515-521; Jang et al., 1995 Circulation 92: 3041-3050).

Fibrinolytic Agents

Fibrinolysis is a naturally occurring process that removes unneeded clots once healing has occurred. The critical step in this system is the transformation of plasminogen into plasmin, a protein-digesting enzyme. Plasmin dissolves thrombus by lysing fibrin.

Fibrinolytic drugs promote the formation of plasmin. Examples of fibrinolytic agents include, but are not limited to, tissue plasminogen activator, urokinase, streptokinase, staphylokinase, anistreplase, reteplase, lanoteplase (Valji, 2000 JVIR 11: 411-420) retinoids such as all-trans-retinoic acid.

Fibrinolysis activity of agents can be assayed by monitoring the dissolution of thrombus labeled with radioactive fibrin (Herbert et al., 1993 Thrombosis and Haemostasis 69: 268-271).

Cell Cycle Inhibitors.

Briefly, a wide variety of cell cycle inhibitory agents can be utilized, either with or without a carrier (e.g., a polymer or ointment or vector), in order to treat or prevent a hyperproliferative disease. Representative examples of such agents include taxanes (e.g., paclitaxel (discussed in more detail below) and docetaxel) (Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(40):351-386, 1993), Etanidazole, Nimorazole (B. A. Chabner and D. L. Longo. Cancer Chemotherapy and Biotherapy—Principles and Practice. Lippincott-Raven Publishers, New York, 1996, p.554), perfluorochemicals with hyperbaric oxygen, transfusion, erythropoietin, BW12C, nicotinamide, hydralazine, BSO, WR-2721, IudR, DUdR, etanidazole, WR-2721, BSO, mono-substituted keto-aldehyde compounds (L. G. Egyud. Keto-aldehyde-amine addition products and method of making same. U.S. Pat. No. 4,066,650, Jan. 3, 1978), nitroimidazole (K. C. Agrawal and M. Sakaguchi. Nitroimidazole radiosensitizers for Hypoxic tumor cells and compositions thereof. U.S. Pat. No. 4,462,992, Jul. 31, 1984), 5-substituted-4-nitroimidazoles (Adams et al., Int. J. Radiat. Biol. Relat. Stud. Phys., Chem. Med. 40(2):153-61, 1981), SR-2508 (Brown et al., Int. J. Radiat. Oncol., Biol. Phys. 7(6):695-703, 1981), 2H-isoindolediones (J. A. Myers, 2H-Isoindolediones, their synthesis and use as radiosensitizers. U.S. Pat. No. 4,494,547, Jan. 22, 1985), chiral [[(2-bromoethyl)-amino]methyl]-nitro-1H-imidazole-1-ethanol (V. G. Beylin, et al., Process for preparing chiral [[(2-bromoethyl)-amino]methyl]-nitro-1H-imidazole-1-ethanol and related compounds. U.S. Pat. No. 5,543,527, Aug. 6, 1996; U.S. Pat. No. 4,797,397; Jan. 10, 1989; U.S. Pat. No. 5,342,959, Aug. 30, 1994), nitroaniline derivatives (W. A. Denny, et al. Nitroaniline derivatives and their use as anti-tumor agents. U.S. Pat. No. 5,571,845, Nov. 5, 1996), DNA-affinic hypoxia selective cytotoxins (M. V. Papadopoulou-Rosenzweig. DNA-affinic hypoxia selective cytotoxins. U.S. Pat. No. 5,602,142, Feb. 11, 1997), halogenated DNA ligand (R. F. Martin. Halogenated DNA ligand radiosensitizers for cancer therapy. U.S. Pat. No. 5,641,764, Jun. 24, 1997), 1,2,4 benzotriazine oxides (W.W. Lee et al. 1,2,4-benzotriazine oxides as radiosensitizers and selective cytotoxic agents. U.S. Pat. No. 5,616,584, Apr. 1, 1997; U.S. Pat. No. 5,624,925, Apr. 29, 1997; Process-for Preparing 1,2,4 Benzotriazine oxides. U.S. Pat. No. 5,175,287, Dec. 29, 1992), nitric oxide (J. B. Mitchell et al., Use of Nitric oxide releasing compounds as hypoxic cell radiation sensitizers. U.S. Pat. No. 5,650,442, Jul. 22, 1997), 2-nitroimidazole derivatives (M. J. Suto et al. 2-Nitroimidazole derivatives useful as radiosensitizers for hypoxic tumor cells. U.S. Pat. No. 4,797,397, Jan. 10, 1989; T. Suzuki. 2-Nitroimidazole derivative, production thereof, and radiosensitizer containing the same as active ingredient. U.S. Pat. No. 5,270,330, Dec. 14, 1993; T. Suzuki et al. 2-Nitroimidazole derivative, production thereof, and radiosensitizer containing the same as active ingredient. U.S. Pat. No. 5,270,330, Dec. 14, 1993; T. Suzuki. 2-Nitroimidazole derivative, production thereof and radiosensitizer containing the same as active ingredient; Patent EP 0 513 351 B1, Jan. 24, 1991), fluorine-containing nitroazole derivatives (T. Kagiya. Fluorine-containing nitroazole derivatives and radiosensitizer comprising the same. U.S. Pat. No. 4,927,941, May 22, 1990), copper (M. J. Abrams. Copper Radiosensitizers. U.S. Pat. No. 5,100,885, Mar. 31, 1992), combination modality cancer therapy (D. H. Picker et al. Combination modality cancer therapy. U.S. Pat. No. 4,681,091, Jul. 21, 1987). 5-CldC or (d)H₄U or 5-halo-2′-halo-2′-deoxy-cytidine or -uridine derivatives (S. B. Greer. Method and Materials for sensitizing neoplastic tissue to radiation. U.S. Pat. No. 4,894,364 Jan. 16, 1990), platinum complexes (K. A. Skov. Platinum Complexes with one radiosensitizing ligand. U.S. Pat. No. 4,921,963. May 1, 1990; K. A. Skov. Platinum Complexes with one radiosensitizing ligand. Patent EP 0 287 317 A3), fluorine-containing nitroazole (T. Kagiya, et al. Fluorine-containing nitroazole derivatives and radiosensitizer comprising the same. U.S. Pat. No. 4,927,941. May 22, 1990), benzamide (W. W. Lee. Substituted Benzamide Radiosensitizers. U.S. Pat. No. 5,032,617, Jul. 16, 1991), autobiotics (L. G. Egyud. Autobiotics and their use in eliminating nonself cells in vivo. U.S. Pat. No. 5,147,652. Sep. 15, 1992), benzamide and nicotinamide (W. W. Lee et al. Benzamide and Nictoinamide Radiosensitizers. U.S. Pat. No. 5,215,738, Jun. 1, 1993), acridine-intercalator (M. Papadopoulou-Rosenzweig. Acridine Intercalator based hypoxia selective cytotoxins. U.S. Pat. No. 5,294,715, Mar. 15, 1994), fluorine-containing nitroimidazole (T. Kagiya et al. Fluorine containing nitroimidazole compounds. U.S. Pat. No. 5,304,654, Apr. 19, 1994), hydroxylated texaphyrins (J. L. Sessler et al. Hydroxylated texaphrins. U.S. Pat. No. 5,457,183, Oct. 10, 1995), hydroxylated compound derivative (T. Suzuki et al. Heterocyclic compound derivative, production thereof and radiosensitizer and antiviral agent containing said derivative as active ingredient. Publication Number 011106775 A (Japan), Oct. 22, 1987; T. Suzuki et al. Heterocyclic compound derivative, production thereof and radiosensitizer, antiviral agent and anti cancer agent containing said derivative as active ingredient. Publication Number 01139596 A (Japan), Nov. 25, 1987; S. Sakaguchi et al Heterocyclic compound derivative, its production and radiosensitizer containing said derivative as active ingredient; Publication Number 63170375 A (Japan), Jan. 7, 1987), fluorine containing 3-nitro-1,2,4-triazole (T. Kagitani et al. Novel fluorine-containing 3-nitro-1,2,4-triazole and radiosensitizer containing same compound. Publication Number 02076861 A (Japan), Mar. 31, 1988), 5-thiotretrazole derivative or its salt (E. Kano et al. Radiosensitizer for Hypoxic cell. Publication Number 61010511 A (Japan), Jun. 26, 1984), Nitrothiazole (T Kagitani et al. Radiation-sensitizing agent. Publication Number 61167616 A (Japan) Jan. 22, 1985), imidazole derivatives (S. Inayma et al. Imidazole derivative. Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (T. Kagitani et al. Radiosensitizer. Publication Number 62039525 A (Japan), Aug. 15, 1985), 3-nitro-1,2,4-triazole (T. Kagitani et al. Radiosensitizer. Publication Number 62138427 A (Japan), Dec. 12, 1985), Carcinostatic action regulator (H. Amagase. Carcinostatic action regulator. Publication Number 63099017 A (Japan), Nov. 21, 1986), 4,5-dinitroimidazole derivative (S. Inayama. 4,5-Dinitroimidazole derivative. Publication Number 63310873 A (Japan) Jun. 9, 1987), nitrotriazole Compound (T. Kagitanil. Nitrotriazole Compound. Publication Number 07149737 A (Japan) Jun. 22, 1993), cisplatin, doxorubin, misonidazole, mitomycin, tiripazamine, nitrosourea, mercaptopurine, methotrexate, flurouracil, bleomycin, vincristine, carboplatin, epirubicin, doxorubicin, cyclophosphamide, vindesine, etoposide (I. F. Tannock. Review Article: Treatment of Cancer with Radiation and Drugs. Journal of Clinical Oncology 14(12):3156-3174, 1996), camptothecin (Ewend M. G. et al. Local delivery of chemotherapy and concurrent external beam radiotherapy prolongs survival in metastatic brain tumor models. Cancer Research 56(22):5217-5223, 1996) and paclitaxel (Tishler R. B. et al. Taxol: a novel radiation sensitizer. International Journal of Radiation Oncology and Biological Physics 22(3):613-617, 1992).

A number of the above-mentioned cell cycle inhibitors also have a wide variety of analogues and derivatives, including, but not limited to, cisplatin, cyclophosphamide, misonidazole, tiripazamine, nitrosourea, mercaptopurine, methotrexate, flurouracil, epirubicin, doxorubicin, vindesine and etoposide. Analogues and derivatives include (CPA)₂Pt[DOLYM] and (DACH)Pt[DOLYM] cisplatin (Choi et al., Arch. Pharmacal Res. 22(2):151-156, 1999), Cis-[PtCl₂(4,7-H-5-methyl-7-oxo]1,2,4[triazolo[1,5-a]pyrimidine)₂] (Navarro et al., J. Med. Chem. 41(3):332-338, 1998), [Pt(cis-1,4-DACH)(trans-Cl₂)(CBDCA)].½MeOH cisplatin (Shamsuddin et al., Inorg. Chem. 36(25):5969-5971, 1997), 4-pyridoxate diammine hydroxy platinum (Tokunaga et al., Pharm. Sci. 3(7):353-356, 1997), Pt(II) . . . . Pt(II) (Pt₂[NHCHN(C(CH₂)(CH₃))]₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans, cis-[Pt(OAc)₂₁₂(en)] (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-[Pt(NH₃)(4-aminoTEMP-O){d(GpG)}] (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23, 1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), CI-973 cisplatin analogue (Yang et al., Int. J. Oncol. 5(3):597-602, 1994), cis-diamminedichloroplatinum(II) and its analogues cis-1,1-cyclobutanedicarbosylato(2R)-2-methyl-1,4-butanediam-mineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bemays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al, Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexanemalonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57, 1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother Oncol. 9(2):157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et al., J. Chem. Soc., Chem. Commun. 6:443-5, 1987), aliphatic tricarboxylic acid platinum complexes (EPA 185225), cis-dichloro(amino acid)(tert-butylamine)platinum(II) complexes (Pasini & Bersanetti, Inorg. Chim. Acta 107(4):259-67, 1985); 4-hydroperoxycylcophosphamide (Ballard et al., Cancer Chemother Pharmacol. 26(6):397-402, 1990), acyclouridine cyclophosphamide derivatives (Zakerinia et al., Helv. Chim. Acta 73(4):912-15, 1990), 1,3,2-dioxa- and -oxazaphosphorinane cyclophosphamide analogues (Yang et al., Tetrahedron 44(20):6305-14, 1988), C5-substituted cyclophosphamide analogues (Spada, University of Rhode Island Dissertation, 1987), tetrahydrooxazine cyclophosphamide analogues (Valente, University of Rochester Dissertation, 1988), phenyl ketone cyclophosphamide analogues (Hales et al., Teratology 39(1):31-7, 1989), phenylketophosphamide cyclophosphamide analogues (Ludeman et al., J. Med. Chem. 29(5):716-27, 1986), ASTA Z-7557 cyclophosphamide analogues (Evans et al., Int. J. Cancer 34(6):883-90, 1984), 3-(1-oxy-2,2,6,6-tetramethyl-4-piperidinyl)cyclophosphamide (Tsui et al., J. Med. Chem. 25(9):1106-10, 1982), 2-oxobis(2-β-chloroethylamino)-4-,6-dimethyl-1,3,2-oxazaphosphorinane cyclophosphamide (Carpenter et al., Phosphorus Sulfur 12(3):287-93, 1982), 5-fluoro- and 5-chlorocyclophosphamide (Foster et al., J. Med. Chem. 24(12):1399-403, 1981), cis- and trans-4-phenylcyclophosphamide (Boyd et al., J. Med. Chem. 23(4):372-5, 1980), 5-bromocyclophosphamide, 3,5-dehydrocyclophosphamide (Ludeman et al., J. Med. Chem. 22(2):151-8, 1979), 4-ethoxycarbonyl cyclophosphamide analogues (Foster, J. Pharm. Sci. 67(5):709-10, 1978), arylaminotetrahydro-2H-1,3,2-oxazaphosphorine 2-oxide cyclophosphamide analogues (Hamacher, Arch. Pharm. (Weinheim, Ger.) 310(5):J,428-34, 1977), NSC-26271 cyclophosphamide analogues (Montgomery & Struck, Cancer Treat. Rep. 60(4):J381-93, 1976), benzo annulated cyclophosphamide analogues (Ludeman & Zon, J. Med. Chem. 18(12):J1251-3, 1975), 6-trifluoromethylcyclophosphamide (Farmer & Cox, J. Med. Chem. 18(11):J1106-10, 1975), 4-methylcyclophosphamide and 6-methycyclophosphamide analogues (Cox et al., Biochem. Pharmacol. 24(5):J599-606, 1975); FCE 23762 doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839, 1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Nat'l Acad. Sci. U.S.A. 95(4):1794-1799, 1998), disaccharide doxorubicin analogues (Arcamone et al., J. Nat'l Cancer Inst. 89(16):1217-1223, 1997), 4-demethoxy-7-O-[2,6-dideoxy-4-O-(2,3,6-trideoxy-3-amino-α-L-lyxo-hexopyranosyl)-α-L-lyxo-hexopyranosyl]-adriamicinone doxorubicin disaccharide analog (Monteagudo et al., Carbohydr Res. 300(1):11-16, 1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int. J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90, 1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9, 1990), 4′-epidoxorubicin (Drzewoski et al., Pol. J. Pharmacol. Pharm. 40(2):159-65, 1988; Weenen et al., Eur. J. Cancer Clin. Oncol. 20(7):919-26, 1984), alkylating cyanomorpholino doxorubicin derivative (Scudder et al., J. Nat'l Cancer Inst. 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoelzel et al., Leuk. Res. 10(12):1455-9, 1986), 4-demethyoxy-4′-o-methyldoxorubicin (Giuliani et al., Proc. Int. Congr. Chemother 16:285-70-285-77, 1983), 3′-deamino-3′-hydroxydoxorubicin (Horton et al., J. Antibiot. 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20, 1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole (Born et al., Biochem. Pharmacol. 43(6):1337-44, 1992), azo and azoxy misonidazole derivatives (Gattavecchia & Tonelli, Int. J. Radiat. Biol. Relat. Stud. Phys., Chem. Med. 45(5):469-77, 1984); RB90740 (Wardman et al., Br. J. Cancer, 74 Suppl. (27):S70-S74, 1996); 6-bromo and 6-chloro-2,3-dihydro-1,4-benzothiazines nitrosourea derivatives (Rai et al., Heterocycl. Commun. 2(6):587-592, 1996), diamino acid nitrosourea derivatives (Dulude et al., Bioorg. Med Chem. Lett. 4(22):2697-700, 1994; Dulude et al., Bioorg. Med. Chem. 3(2):151-60, 1995), amino acid nitrosourea derivatives (Zheleva et al., Pharmazie 50(1):25-6, 1995), 3′,4′-didemethoxy-3′,4′-dioxo-4-deoxypodophyllotoxin nitrosourea derivatives (Miyahara et al., Heterocycles 39(1):361-9, 1994), ACNU (Matsunaga et al., Immunopharmacology 23(3):199-204, 1992), tertiary phosphine oxide nitrosourea derivatives (Guguva et al., Pharmazie 46(8):603, 1991), sulfamerizine and sulfamethizole nitrosourea derivatives (Chiang et al., Zhonghua Yaozue Zazhi 43(5):401-6, 1991), thymidine nitrosourea analogues (Zhang et al., Cancer Commun. 3(4):119-26, 1991), 1,3-bis(2-chloroethyl)-1-nitrosourea (August et al., Cancer Res. 51(6):1586-90, 1991), 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine (Boutin et al., Eur. J. Cancer Clin. Oncol. 25(9):1311-16, 1989), pyrimidine (II) nitrosourea derivatives (Wei et al., Chung-hua Yao Hsueh Tsa Chih 41(1):19-26, 1989), CGP 6809 (Schieweck et al., Cancer Chemother Pharmacol. 23(6):341-7, 1989), B-3839 (Prajda et al., In Vivo 2(2):151-4, 1988), 5-halogenocytosine nitrosourea derivatives (Chiang & Tseng, T'ai-wan Yao Hsueh Tsa Chih 38(1):37-43, 1986), 1-(2-chloroethyl)-3-isobutyl-3-(β-maltosyl)-1-nitrosourea (Fujimoto & Ogawa, J. Pharmacobio-Dyn. 10(7):341-5, 1987), sulfur-containing nitrosoureas (Tang et al., Yaoxue Xuebao 21(7):502-9, 1986), sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives (Tanoh et al., Chemotherapy (Tokyo) 33(11):969-77, 1985), CNCC, RFCNU and chlorozotocin (Mena et al., Chemotherapy (Basel) 32(2):131-7, 1986), CNUA (Edanami et al., Chemotherapy (Tokyo) 33(5):455-61, 1985), 1-(2-chloroethyl)-3-isobutyl-3-(β-maltosyl)-1-nitrosourea (Fujimoto & Ogawa, Jpn. J. Cancer Res. (Gann) 76(7):651-6, 1985), choline-like nitrosoalkylureas (Belyaev et al., Izv. Akad. NAUK SSSR, Ser Khim. 3:553-7, 1985), sucrose nitrosourea derivatives (JP 84219300), sulfa drug nitrosourea analogues (Chiang et al., Proc. Nat'l Sci. Counc., Repub. China, Part A 8(1):18-22, 1984), DONU (Asanuma et al., J. Jpn. Soc. Cancer Ther. 17(8):2035-43, 1982), N,N′-bis (N-(2-chloroethyl)-N-nitrosocarbamoyl)cystamine (CNCC) (Blazsek et al., Toxicol. Appl. Pharmacol. 74(2):250-7, 1984), dimethylnitrosourea (Krutova et al., Izv. Akad. NAUK SSSR, Ser Biol. 3:439-45, 1984), GANU (Sava & Giraldi, Cancer Chemother Pharmacol. 10(3):167-9, 1983), CCNU (Capelli et al., Med., Biol., Environ. 11(1):111-16, 1983), 5-aminomethyl-2′-deoxyuridine nitrosourea analogues (Shiau, Shih Ta Hsueh Pao (Taipei) 27:681-9, 1982), TA-077 (Fujimoto & Ogawa, Cancer Chemother. Pharmacol. 9(3):134-9, 1982), gentianose nitrosourea derivatives (JP 82 80396), CNCC, RFCNU, RPCNU AND chlorozotocin (CZT) (Marzin et al., INSERM Symp., 19(Nitrosoureas Cancer Treat.):165-74, 1981), thiocolchicine nitrosourea analogues (George, Shih Ta Hsueh Pao (Taipei) 25:355-62, 1980), 2-chloroethyl-nitrosourea (Zeller & Eisenbrand, Oncology 38(1):39-42, 1981), ACNU, (1-(4-amino-2-methyl-5-pyrimidinyl)methyl-3-(2-chloroethyl)-3-nitrosourea hydrochloride) (Shibuya et al., Gan To Kagaku Ryoho 7(8):1393-401, 1980), N-deacetylmethyl thiocolchicine nitrosourea analogues (Lin et al., J. Med. Chem. 23(12):1440-2, 1980), pyridine and piperidine nitrosourea derivatives (Crider et al., J. Med. Chem. 23(8):848-51, 1980), methyl-CCNU (Zimber & Perk, Refu. Vet. 35(1):28, 1978), phensuzimide nitrosourea derivatives (Crider et al., J. Med. Chem. 23(3):324-6, 1980), ergoline nitrosourea derivatives (Crider et al., J. Med. Chem. 22(1):32-5, 1979), glucopyranose nitrosourea derivatives (JP 78 95917), 1-(2-chloroethyl)-3-cyclohexyl-1-nitrosourea (Farmer et al., J. Med. Chem. 21(6):514-20, 1978), 4-(3-(2-chloroethyl)-3-nitrosoureid-o)-cis-cyclohexanecarboxylic acid (Drewinko et al., Cancer Treat. Rep. 61(8):J1513-18, 1977), RPCNU (ICIG 1163) (Larnicol et al., Biomedicine 26(3):J176-81, 1977), IOB-252 (Sorodoc et al., Rev. Roum. Med. Virol. 28(1):J55-61, 1977), 1,3-bis(2-chloroethyl)-1-nitrosourea (BCNU) (Siebert & Eisenbrand, Mutat. Res. 42(1):J45-50, 1977), 1-tetrahydroxycyclopentyl-3-nitroso-3-(2-chloroethyl)-urea (U.S. Pat. No. 4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67, 1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7, 1981); indoline ring and a modified omithine or glutamic acid-bearing methotrexate derivatives (Matsuoka et al., Chem. Pharm. Bull. 45(7):1146-1150, 1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaminopterin analogues (DeGraw et al., J. Med. Chem. 40(3):370-376, 1997), 5-deazaminopterin and 5,10-dideazaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm., Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8, 1995), N-(α-aminoacyl) methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), β,γ-methano methotrexate analogues (Rosowsky et al., Pteridines 2(3):133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl) methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No. 4,725,687), Nδ-acyl-Nα-(4-amino-4-deoxypteroyl)-L-omithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed Polym.): 311-24, 1987), methotrexate-γ-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8, 1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol. Clin. Aspects: 989-92, 1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62, 1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9, 1986), 2,.omega.-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122(Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (U.S. Pat. No. 4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.—Chim. Ther. 19(3):267-73, 1984), poly (L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220); N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9, 1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al, Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluyl-5-fluorouracil (Okada, Hiroshima J. Med. Sci. 28(1):49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin (Lanius, Adv. Chemother. Gastrointest. Cancer, (Int. Symp.), 159-67, 1984); N-substituted deacetylvinblastine amide (vindesine) sulfates (Conrad et al., J. Med. Chem. 22(4):391-400, 1979); and Cu(II)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Lett. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

Within one preferred embodiment of the invention, the cell cycle inhibitor is taxane such as paclitaxel. Briefly, taxanes are compounds which disrupts mitosis (M-phase) by binding to tubulin to form abnormal mitotic spindles or an analogue or derivative thereof. Paclitaxel, the most recognized member of the taxane family is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216, 1993). “Paclitaxel” (which should be understood herein to include formulations, prodrugs, analogues and derivatives such as, for example, TAXOL®, TAXOTERE®, docetaxel, 10-desacetyl analogues of paclitaxel and 3′N-desbenzoyl-3′N-t-butoxy carbonyl analogues of paclitaxel) may be readily prepared utilizing techniques known to those skilled in the art (see, e.g., Schiff et al., Nature 277:665-667, 1979; Long and Fairchild, Cancer Research 54:4355-4361, 1994; Ringel and Horwitz, J. Nat'l Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′-and/or 7-O-ester derivatives), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-γ-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′,7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogs bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the Cell Cycle Inhibitor is a taxane having the formula (C1):

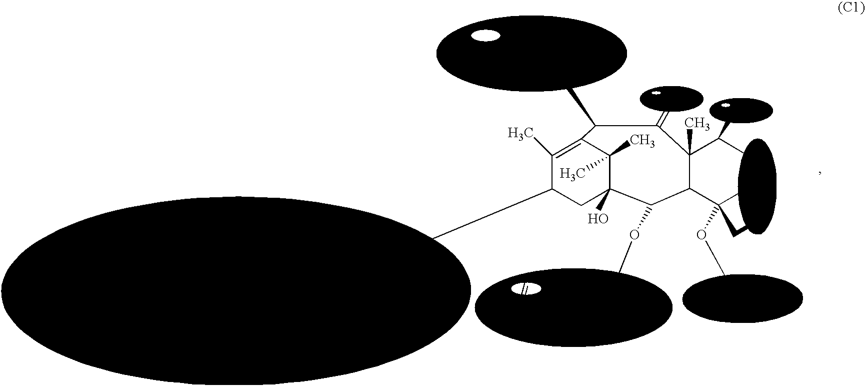

where the gray-highlighted portions may be substituted and the non-highlighted portion is the taxane core. A side-chain (labeled “A” in the diagram) is desirably present in order for the compound to have good activity as a Cell Cycle Inhibitor. Examples of compounds having this structure include paclitaxel (Merck Index entry 7117), docetaxol (Taxotere, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogs and derivatives are disclosed in Patent No. 5,440,056 as having the structure (C2):

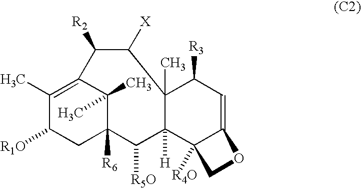

wherein X may be oxygen (paclitaxel), hydrogen (9-deoxy derivatives), thioacyl, or dihydroxyl precursors; R₁ is selected from paclitaxel or taxotere side chains or alkanoyl of the formula (C3)

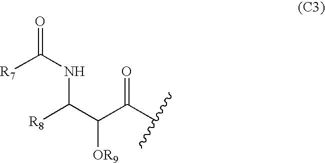

wherein R₇ is selected from hydrogen, alkyl, phenyl, alkoxy, amino, phenoxy (substituted or unsubstituted); R₈ is selected from hydorgen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogs and derivatives useful as Cell Cycle Inhibitors in the present invention are disclosed in PCT International Patent Application No. WO 93/10076. As disclosed in this publication, the analog or derivative should have a side chain attached to the taxane nucleus at C₁₃, as shown in the structure below (formula C4), in order to confer antitumor activity to the taxane.

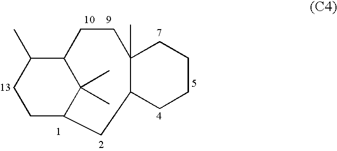

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, 10. As well, an oxetane ring may be attached at carbons 4 and 5. As well, an oxirane ring may be attached to the carbon labeled 4.

In one aspect, the taxane-based Cell Cycle Inhibitor useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel is particular, is considered to function as a Cell Cycle Inhibitor by acting as a anti-microtuble agent, and more specifically as a stabilizer.

In another aspect, the Cell Cycle Inhibitor is a Vinca Alkaloid. Vinca alkaloids have the following general structure. They are indole-dihydroindole dimers.

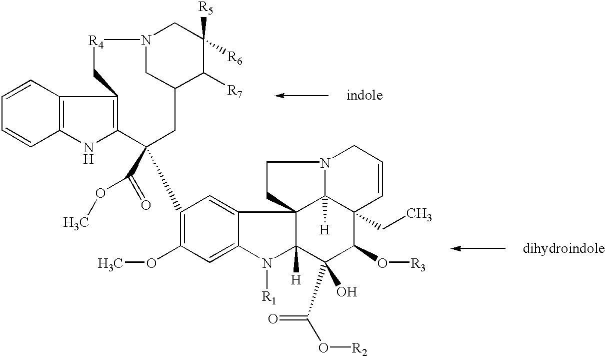

As disclosed in U.S. Pat. Nos. 4,841,045 and 5,030,620, R₁ can be a formyl or methyl group or alternately H. R₁ could also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately, a protein fragment may be linked by a bifunctional group such as maleoyl amino acid. R₃ could also be substituted to form an alkyl ester which may be further substituted. R₄ may be —CH₂— or a single bond. R₅ and R₆ may be H, OH or a lower alkyl, typically —CH₂CH₃. Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary Vinca Alkaloids are vinblastine, vincristine, vincristine sulfate, vindesine, and vinorelbine, having the structures:

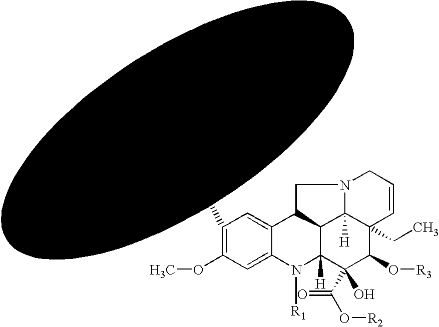

| | R₁ | R₂ | R₃ | R₄ | R₅ |
|---|---|---|---|---|---|
| Vinblastine: | CH₃ | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₃ | CH₃ | H | single bond |

Analogs typically require the side group (shaded area) in order to have activity. These compounds are believed to act as Cell Cycle Inhibitors by functioning as anti-microtubule agents, and more specifically to inhibit polymerization.

In another aspect, the Cell Cycle Inhibitor is Camptothecin, or an analog or derivative thereof. Camptothecins have the following general structure.

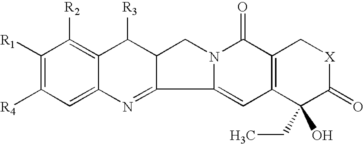

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

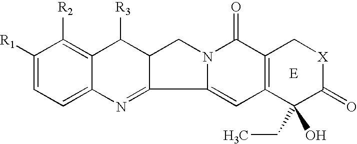

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity. These compounds are useful to as Cell Cycle Inhibitors, where they function as Topoisomerase I Inhibitors and/or DNA cleavage agents.

In another aspect, the Cell Cycle Inhibitor is a Podophyllotoxin, or a derivative or an analog thereof. Exemplary compounds of this type are Etoposide or Teniposide, which have the following structures:

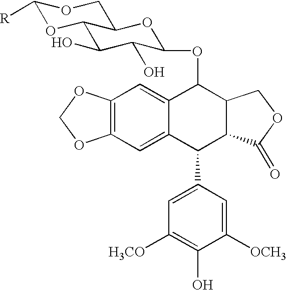

| | R |
|---|---|
| Etoposide | CH₃ |
| Teniposide | S |

These compounds are believed to function as Cell Cycle Inhibitors by being Topoisomerase II Inhibitors and/or by DNA cleaving agents.

In another aspect, the Cell Cycle Inhibitor is an Anthracycline. Anthracyclines have the following general structure, where the R groups may be a variety of organic groups:

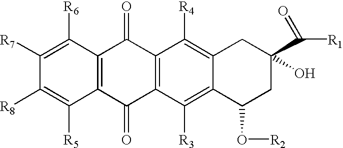

According to U.S. Pat. No. 5,594,158, suitable R groups are: R₁ is CH₃ or CH₂OH; R₂ is daunosamine or H; R₃ and R₄ are independently one of OH, NO₂, NH₂, F, Cl, Br, I, CN, H or groups derived from these; R₅₋₇ are all H or R₅ and R₆ are H and R₇ and R₈ are alkyl or halogen, or vice versa: R₇ and R₈ are H and R₅ and R₆ are alkyl or halogen.

According to U.S. Pat. No. 5,843,903, R₂ may be a conjugated peptide. According to U.S. Pat. Nos. 4,215,062 and 4,296,105, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure:

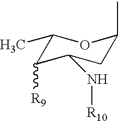

in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary Anthracycline are Doxorubicin, Daunorubicin, Idarubicin, Epirubicin, Pirarubicin, Zorubicin, and Carubicin. Suitable compounds have the structures:

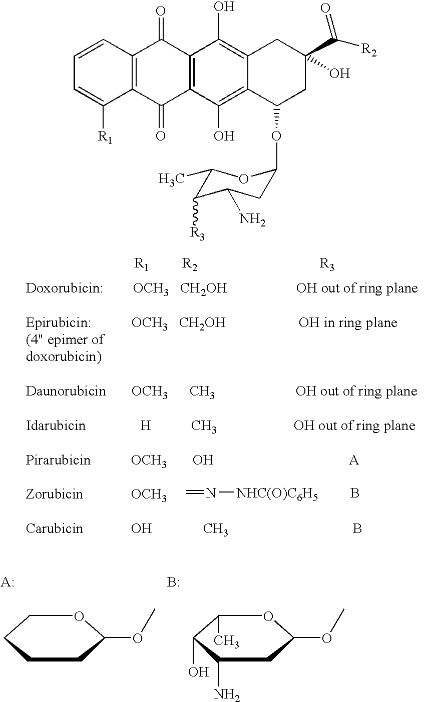

Other suitable Anthracyclines are Anthramycin, Mitoxantrone, Menogaril, Nogalamycin, Aclacinomycin A, Olivomycin A, Chromomycin A₃, and Plicamycin having the structures:

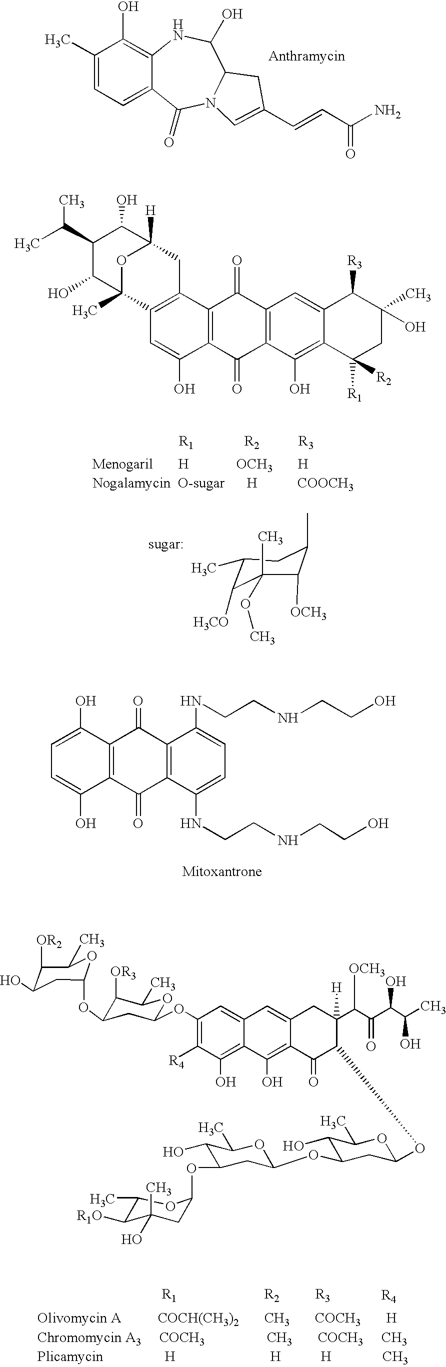

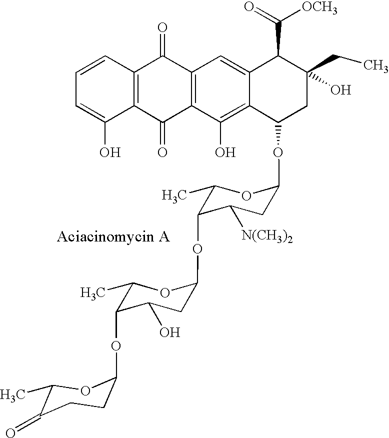

These compounds are believed to function as Cell Cycle Inhibitors by being Topoisomerase Inhibitors and/or by DNA cleaving agents.

In another aspect, the Cell Cycle Inhibitor is a Platinum compound. In general, suitable platinum complexes may be of Pt(II) or Pt(IV) and have this basic structure:

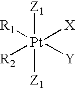

wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

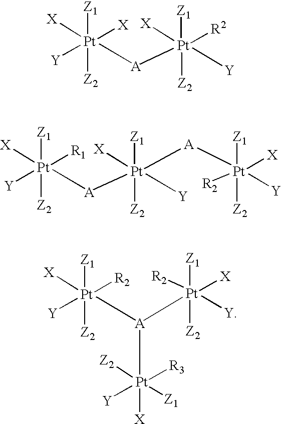

Exemplary Platinum compound are Cisplatin, Carboplatin, Oxaliplatin, and Miboplatin having the structures:

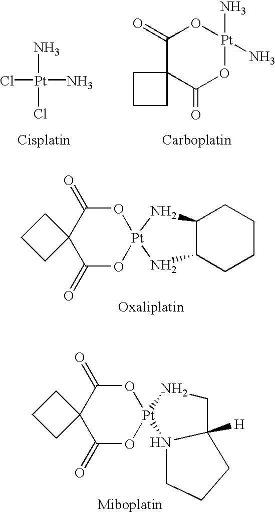

These compounds are believed to function as Cell Cycle Inhibitors by binding to DNA, i.e., acting as alkylating agents of DNA.

In another aspect, the Cell Cycle Inhibitor is a Nitrosourea. Nitrosourease have the following general structure (C5), where typical R groups are shown below.

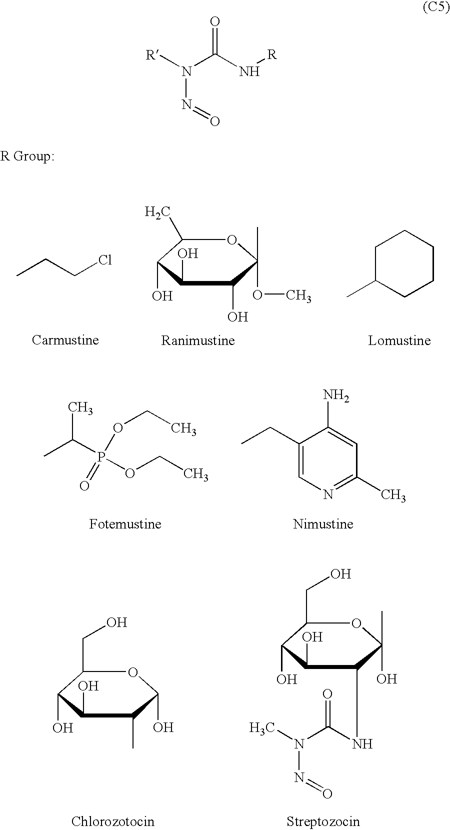

Other suitable R groups include cyclic alkanes, alkanes, halogen substituted groups, sugars, aryl and heteroaryl groups, phosphonyl and sulfonyl groups. As disclosed in U.S. Pat. No. 4,367,239, R may suitably be CH₂—C(X)(Y)(Z), wherein X and Y may be the same or different members of the following groups: phenyl, cyclyhexyl, or a phenyl or cyclohexyl group substituted with groups such as halogen, lower alkyl (C₁₋₄), trifluore methyl, cyano, phenyl, cyclohexyl, lower alkyloxy (C₁₋₄). Z has the following structure: -alkylene-N—R₁R₂, where R₁ and R₂ may be the same or different members of the following group: lower alkyl (C₁₋₄) and benzyl, or together R₁ and R₂ may form a saturated 5 or 6 membered heterocyclic such as pyrrolidine, piperidine, morfoline, thiomorfoline, N-lower alkyl piperazine, where the heterocyclic may be optionally substituted with lower alkyl groups.

As disclosed in U.S. Pat. No. 6,096,923, R and R′ of formula (C5) may be the same or different, where each may be a substituted or unsubstituted hydrocarbon having 1-10 carbons. Substitutions may include hydrocarbyl, halo, ester, amide, carboxylic acid, ether, thioether and alcohol groups. As disclosed in U.S. Pat. No. 4,472,379, R of formula (C5) may be an amide bond and a pyranose structure (e.g. Methyl 2′-[N-[N-(2-chloroethyl)-N-nitroso-carbamoyl]-glycyl]amino-2′-deoxy-α-D-glucopyrano side). As disclosed in U.S. Pat. No. 4,150,146, R of formula (C5) may be an alkyl group of 2 to 6 carbons and may be substituted with an ester, sulfonyl, or hydroxyl group. It may also be substituted with a carboxylica acid or CONH₂ group.

Exemplary Nitrosourea are BCNU (Carmustine), Methyl-CCNU (Semustine), CCNU (Lomustine), Ranimustine, Nimustine, Chlorozotocin, Fotemustine, Streptozocin, and Streptozocin, having the structures:

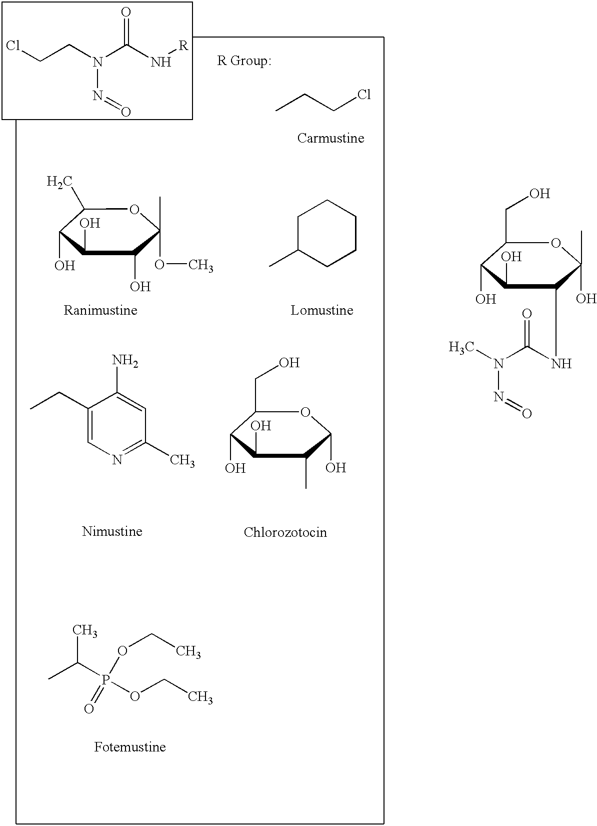

These nitrosourea compounds are believed to function as Cell Cycle Inhibitor by binding to DNA, that is, by functioning as DNA alkylating agents.

In another aspect, the Cell Cycle Inhibitor is a Nitroimidazole, where exemplary Nitroimidazoles are Metronidazole, Benznidazole, Etanidazole, and Misonidazole, having the structures:

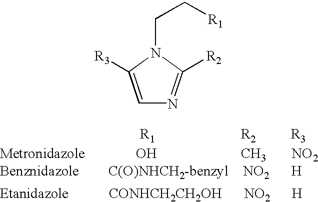

Suitable nitroimidazole compounds are disclosed in, e.g., U.S. Pat. Nos. 4,371,540 and 4,462,992.

In another aspect, the Cell Cycle Inhibitor is a Folic acid antagonist, such as Methotrexate or derivatives or analogs thereof, including Edatrexate, Trimetrexate, Raltitrexed, Piritrexim, Denopterin, Tomudex, and Pteropterin. Methotrexate analogs have the following general structure:

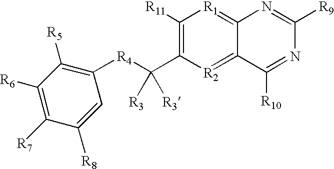

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R₅,₆,₈ may be H, OCH₃, or alternately they can be halogens or hydro groups. R₇ is a side chain of the general structure:

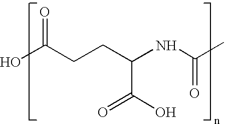

wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

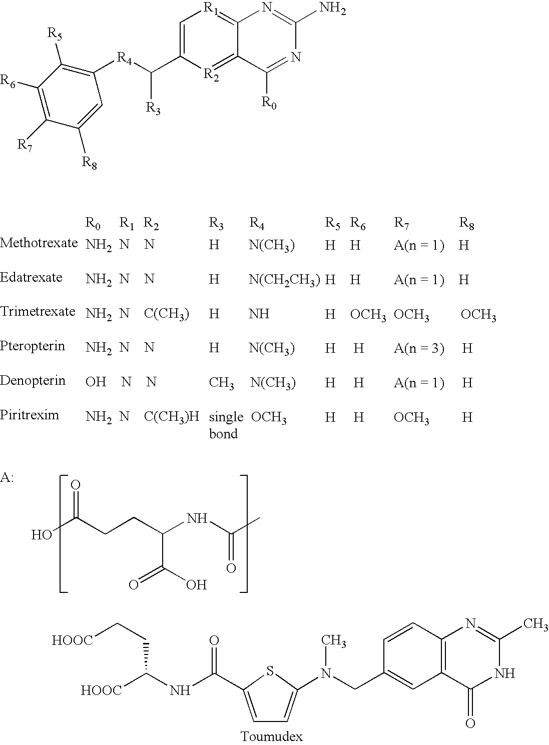

These compounds are believed to function as Cell Cycle Inhibitors by serving as antimetabolites of folic acid.

In another aspect, the Cell Cycle Inhibitor is a Cytidine Analog, such as Cytarabine or derivatives or analogs thereof, including Enocitabine, FMdC ((E(-2′-deoxy-2′-(fluoromethylene)cytidine), Gemcitabine, 5-Azacitidine, Ancitabine, and 6-Azauridine. Exemplary compounds have the structures:

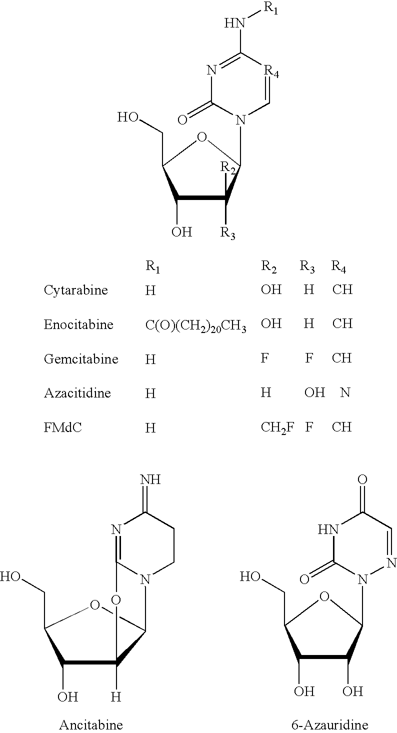

These compounds are believed to function as Cell Cycle Inhibitors as acting as antimetabolites of pyrimidine.

In another aspect, the Cell Cycle Inhibitor is a Pyrimidine analog. In one aspect, the Pyrimidine analogs have the general structure:

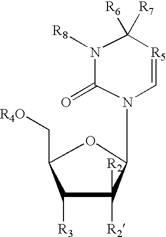

wherein positions 2′, 3′ and 5′ on the sugar ring (R₂, R₃ and R₄, respectively) can be H, hydroxyl, phosphoryl (see, e.g., U.S. Pat. No. 4,086,417) or ester (see, e.g., U.S. Pat. No. 3,894,000). Esters can be of alkyl, cycloalkyl, aryl or heterocyclo/aryl types. The 2′ carbon can be hydroxylated at either R₂ or R₂′, the other group is H. Alternately, the 2′ carbon can be substituted with halogens e.g., fluoro or difluoro cytidines such as Gemcytabine. Alternately, the sugar can be substituted for another heterocyclic group such as a furyl group or for an alkane, an alkyl ether or an amide linked alkane such as C(O)NH(CH₂)₅CH₃. The 2° amine can be substituted with an aliphatic acyl (R₁) linked with an amide (see, e.g., U.S. Pat. No. 3,991,045) or urethane (see, e.g., U.S. Pat. No. 3,894,000) bond. It can also be further substituted to form a quaternary ammonium salt. R₅ in the pyrimidine ring may be N or CR, where R is H, halogen containing groups, or alkyl (see, e.g., U.S. Pat. No. 4,086,417). R₆ and R₇ can together can form an oxo group or R₆=—NH—R₁ and R₇=H. R₈ is H or R₇ and R₈ together can form a double bond or R₈ can be X, where X is:

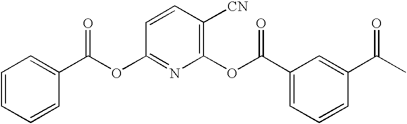

Specific pyrimidine analogs are disclosed in U.S. Pat. No. 3,894,000 (see, e.g., 2′-O-palmityl-ara-cytidine, 3′-O-benzoyl-ara-cytidine, and more than 10 other examples); U.S. Pat. No. 3,991,045 (see, e.g., N4-acyl-1-β-D-arabinofuranosylcytosine, and numerous acyl groups derivatives as listed therein, such as palmitoyl.

In another aspect, the Cell Cycle Inhibitor is a Fluoro-pyrimidine Analog, such as 5-Fluorouracil, or an analog or derivative thereof, including Carmofur, Doxifluridine, Emitefur, Tegafur, and Floxuridine. Exemplary compounds have the structures:

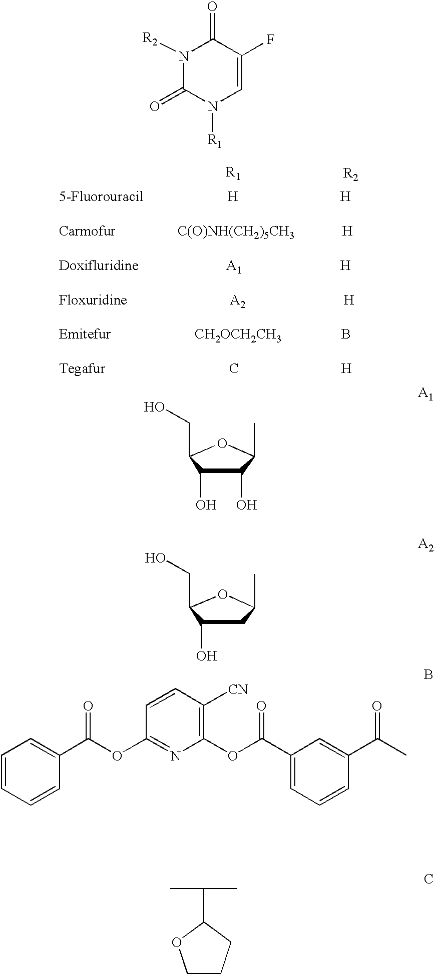

Other suitable Fluoropyrimidine Analogs include 5-FudR (5-fluoro-deoxyuridine), or an analog or derivative thereof, including 5-iododeoxyuridine (5-IudR), 5-bromodeoxyuridine (5-BudR), Fluorouridine triphosphate (5-FUTP), and Fluorodeoxyuridine monophosphate (5-dFUMP). Exemplary compounds have the structures:

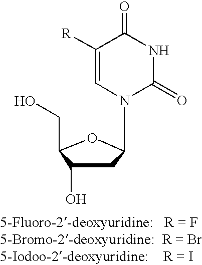

These compounds are believed to function as Cell Cycle Inhibitors by serving as antimetabolites of pyrimidine.

In another aspect, the Cell Cycle Inhibitor is a Purine Analog. Purine analogs have the following general structure:

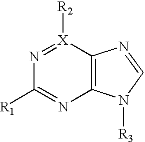

wherein X is typically carbon; R₁ is H, halogen, amine or a substituted phenyl; R₂ is H, a primary, secondary or tertiary amine, a sulfur containing group, typically —SH, an alkane, a cyclic alkane, a heterocyclic or a sugar; R₃ is H, a sugar (typically a furanose or pyranose structure), a substituted sugar or a cyclic or heterocyclic alkane or aryl group. See, e.g., U.S. Pat. No. 5,602,140 for compounds of this type.

In the case of pentostatin, X—R₂ is —CH₂CH(OH)—. In this case a second carbon atom is inserted in the ring between X and the adjacent nitrogen atom. The X—N double bond becomes a single bond.

U.S. Pat. No. 5,446,139 describes suitable purine analogs of the type shown in the following formula:

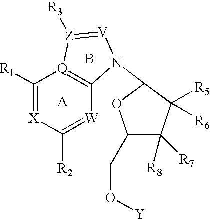

wherein N signifies nitrogen and V, W, X, Z can be either carbon or nitrogen with the following provisos. Ring A may have 0 to 3 nitrogen atoms in its structure. If two nitrogens are present in ring A, one must be in the W position. If only one is present, it must not be in the Q position. V and Q must not be simultaneously nitrogen. Z and Q must not be simultaneously nitrogen. If Z is nitrogen, R₃ is not present. Furthermore, R₁₋₃ are independently one of H, halogen, C₁₋₇ alkyl, C₁₋₇ alkenyl, hydroxyl, mercapto, C₁₋₇ alkylthio, C₁₋₇ alkoxy, C₂₋₇ alkenyloxy, aryl oxy, nitro, primary, secondary or tertiary amine containing group. R₅₋₈ are H or up to two of the positions may contain independently one of OH, halogen, cyano, azido, substituted amino, R₅ and R₇ can together form a double bond. Y is H, a C₁₋₇ alkylcarbonyl, or a mono- di or tri phosphate.

Exemplary suitable purine analogs include 6-Mercaptopurine, Thiguanosine, Thiamiprine, Cladribine, Fludaribine, Tubercidin, Puromycin, Pentoxyfilline; where these compounds may optionally be phosphorylated. Exemplary compounds have the structures:

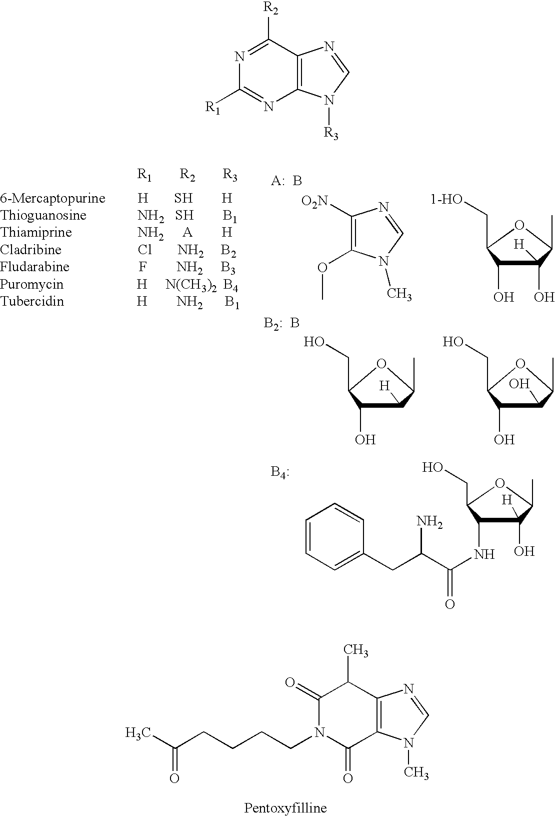

These compounds are believed to function as Cell Cycle Inhibitors by serving as antimetabolites of purine.

In another aspect, the Cell Cycle Inhibitor is a Nitrogen Mustard. Many suitable Nitrogen Mustards are known and are suitably used as a Cell Cycle Inhibitor in the present invention. Suitable nitrogen mustards are also known as cyclophosphamides.

A preferred nitrogen mustard has the general structure:

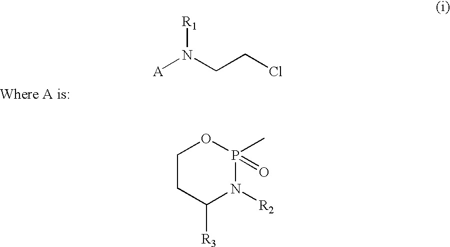

or —CH₃ or other alkane, or chloronated alkane, typically CH₂CH(CH₃)Cl, or a polycyclic group such as B, or a substituted phenyl such as C or a heterocyclic group such as D.

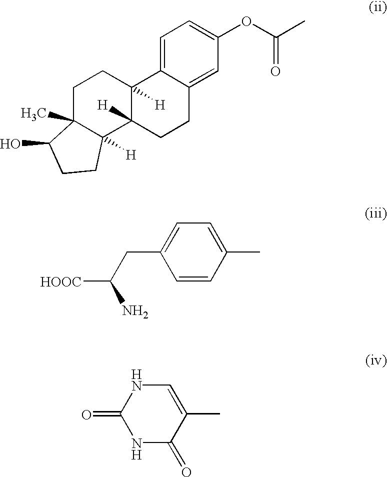

Suitable nitrogen mustards are disclosed in U.S. Pat. No. 3,808,297, wherein A is:

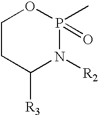

R₁₋₂ are H or CH₂CH₂Cl; R₃ is H or oxygen-containing groups such as hydroperoxy; and R₄ can be alkyl, aryl, heterocyclic.

The cyclic moiety need not be intact. See, e.g., U.S. Pat. Nos. 5,472,956, 4,908,356, 4,841,085 that describe the following type of structure:

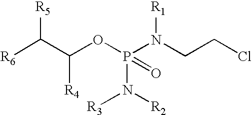

wherein R₁ is H or CH₂CH₂Cl, and R₂₋₆ are various substituent groups.

Exemplary nitrogen mustards include methylchloroethamine, and analogs or derivatives thereof, including methylchloroethamine oxide hydrohchloride, Novembichin, and Mannomustine (a halogenated sugar). Exemplary compounds have the structures:

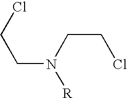

| | R | 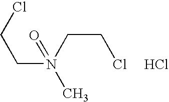 |
|---|---|---|
| Mechlorethanime | CH₃ | Mechlorethanime Oxide HCl |
| Novembichin | CH₂CH(CH₃)Cl | |

The Nitrogen Mustard may be Cyclophosphamide, Ifosfamide, Perfosfamide, or Torofosfamide, where these compounds have the structures:

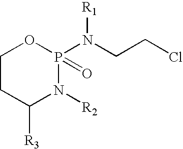

| | R₁ | R₂ | R₃ |
|---|---|---|---|
| Cyclophosphamide | H | CH₂CH₂Cl | H |
| Ifosfamide | CH₂CH₂Cl | H | H |
| Perfosfamide | CH₂CH₂Cl | H | OOH |
| Torofosfamide | CH₂CH₂Cl | CH₂CH₂Cl | H |

The Nitrogen Mustard may be Estramustine, or an analog or derivative thereof, including Phenesterine, Prednimustine, and Estramustine PO₄. Thus, suitable nitrogen mustard type Cell Cycle Inhibitors of the present invention have the structures:

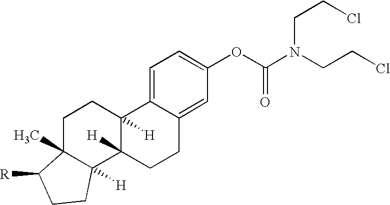

| | R |
|---|---|
| Estramustine | OH |
| Phenesterine | C(CH₃)(CH₂)₃CH(CH₃)₂ |

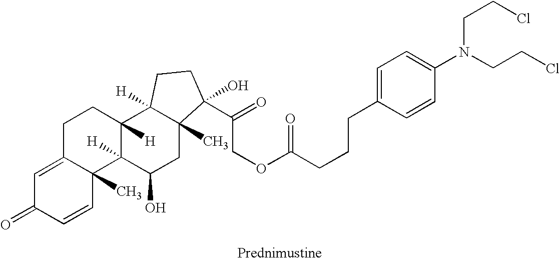

The Nitrogen Mustard may be Chlorambucil, or an analog or derivative thereof, including Melphalan and Chlormaphazine. Thus, suitable nitrogen mustard type Cell Cycle Inhibitors of the present invention have the structures:

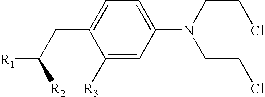

| | R₁ | R₂ | R₃ |
|---|---|---|---|
| Chlorambucil | CH₂COOH | H | H |
| Melphalan | COOH | NH₂ | H |

| | | |
|---|---|---|
| Chlornaphazine | H | together forms a |
| | | benzene ring |

The Nitrogen Mustard may be Uracil Mustard, which has the structure:

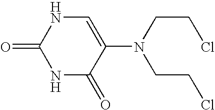

The Nitrogen Mustards are believed to function as Cell Cycle Inhibitors by serving as alkylating agents for DNA. Nitrogen Mustards have been shown useful in the treatment of cell proliferative disorders including, for example, small cell lung, breast, cervical, head and neck, prostate, retinoblastoma, and soft tissue sarcoma.

The Cell Cycle Inhibitor of the present invention may be a Hydroxyurea. Hydroxyureas have the following general structure:

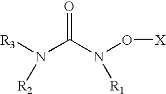

Suitable Hydroxyureas are disclosed in, for example, U.S. Pat. No. 6,080,874, wherein R₁ is:

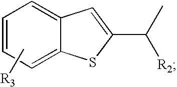

and R₂ is an alkyl group having 1-4 carbons and R₃ is one of H, acyl, methyl, ethyl, and mixtures thereof, such as a methylether.

Other suitable Hydroxyureas are disclosed in, e.g., U.S. Pat. No. 5,665,768, wherein R₁ is a cycloalkenyl group, for example N-[3-[5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable Hydroxyureas are disclosed in, e.g., U.S. Pat. No. 4,299,778, wherein R₁ is a phenyl group substituted with on or more fluorine atoms; R₂ is a cyclopropyl group; and R₃ and X is H.

Other suitable Hydroxyureas are disclosed in, e.g., U.S. Pat. No. 5,066,658, wherein R₂ and R₃ together with the adjacent nitrogen form:

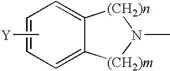

wherein m is 1 or 2, n is 0-2 and Y is an alkyl group.

In one aspect, the hydroxy urea has the structure:

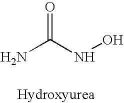

Hydroxyureas are believed to function as Cell Cycle Inhibitors by serving to inhibit DNA synthesis.

In another aspect, the Cell Cycle Inhibitor is a Belomycin, such as Bleomycin A₂, which have the structures:

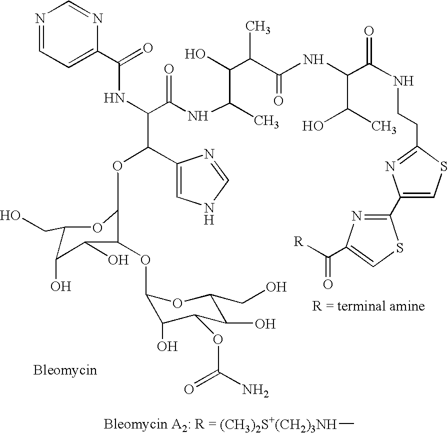

Belomycins are believed to function as Cell Cycle Inhibitors by cleaving DNA.

In another aspect, the Cell Cycle Inhibitor is a Mytomycin, such as Mitomycin C, or an analog or derivative thereof, such as Porphyromycin. Suitable compounds have the structures:

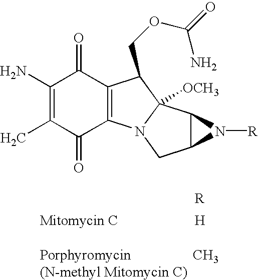

These compounds are believed to function as Cell Cycle Inhibitors by serving as DNA alkylating agents.

In another aspect, the Cell Cycle Inhibitor is an Alkyl sulfonate, such as Busulfan, or an analog or derivative thereof, such as Treosulfan, Improsulfan, Piposulfan, and Pipobroman. Exemplary compounds have the structures:

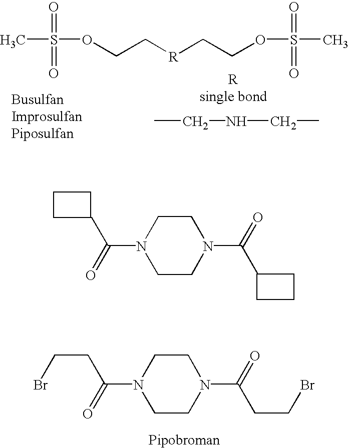

These compounds are believed to function as Cell Cycle Inhibitors by serving as DNA alkylating agents.

In another aspect, the Cell Cycle Inhibitor is a Benzamide. In yet another aspect, the Cell Cycle Inhibitor is a Nicotinamide. These compounds have the basic structure:

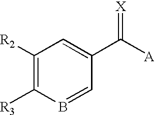

wherein X is either O or S; A is commonly NH₂ or it can be OH or an alkoxy group; B is N or C—R₄, where R₄ is H or an ether-linked hydroxylated alkane such as OCH₂CH₂OH, the alkane may be linear or branched and may contain one or more hydroxyl groups. Alternately, B may be N—R₅ in which case the double bond in the ring involving B is a single bond. R₅ may be H, and alkyl or an aryl group (see, e.g., U.S. Pat. No. 4,258,052); R₂ is H, OR₆, SR₆ or NHR₆, where R₆ is an alkyl group; and R₃ is H, a lower alkyl, an ether linked lower alkyl such as —O-Me or —O-Ethyl (see, e.g., U.S. Pat. No. 5,215,738).

Suitable Benzamide compounds have the structures:

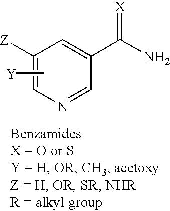

where additional compounds are disclosed in U.S. Pat. No. 5,215,738, (listing some 32 compounds).

Suitable Nicotinamide compounds have the structures:

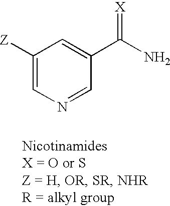

where additional compounds are disclosed in U.S. Pat. No. 5,215,738 (listing some 58 compounds, e.g., 5-OH nicotinamide, 5-aminonicotinamide, 5-(2,3-dihydroxypropoxy) nicotinamide), and compounds having the structures:

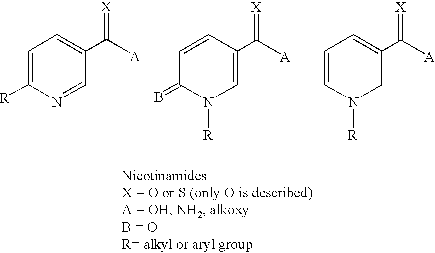

and U.S. Pat. No. 4,258,052 (listing some 46 compounds, e.g., 1-methyl-6-keto-1,6-dihydronicotinic acid).

In one aspect, the Cell Cycle Inhibitor is a Tetrazine Compound, such as Temozolomide, or an analog or derivative thereof, including Dacarbazine. Suitable compounds have the structures:

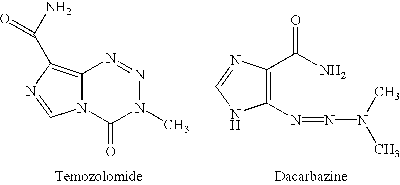

Another suitable Tetrazine Compound is Procarbazine, including HCl and HBr salts, having the structure:

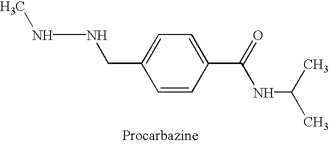

In another aspect, the Cell Cycle Inhibitor is Actinomycin D, or other members of this family, including Dactinomycin, Actinomycin C₁, Actinomycin C₂, Actinomycin C₃, and Actinomycin F₁. Suitable compounds have the structures:

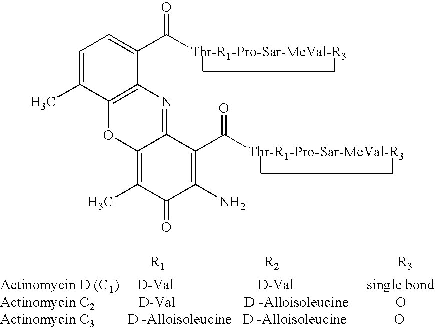

In another aspect, the Cell Cycle Inhibitor is an Aziridine compound, such as Benzodepa, or an analog or derivative thereof, including Meturedepa, Uredepa, and Carboquone. Suitable compounds have the structures:

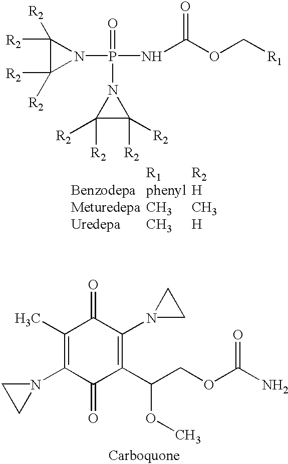

In another aspect, the Cell Cycle Inhibitor is Halogenated Sugar, such as Mitolactol, or an analog or derivative thereof, including Mitobronitol and Mannomustine. Suitable compounds have the structures:

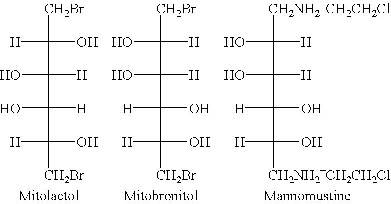

In another aspect, the Cell Cycle Inhibitor is a Diazo compound, such as Azaserine, or an analog or derivative thereof, including 6-diazo-5-oxo-L-norleucine and 5-diazouracil (also a pyrimidine analog). Suitable compounds have the structures:

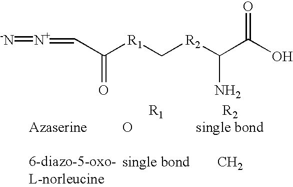

Other compounds that may serve as Cell Cycle Inhibitors according to the present invention are Pazelliptine; Wortmannin; Metoclopramide; RSU; Buthionine sulfoxime; Tumeric; Curcumin; AG337, a thymidylate synthase inhibitor; Levamisole; Lentinan, a polysaccharide; Razoxane, an EDTA analog; Indomethacin; Chlorpromazine; (x and β interferon; MnBOPP; Gadolinium texaphyrin; 4-amino-1,8-naphthalimide; Staurosporine derivative of CGP; and SR-2508.

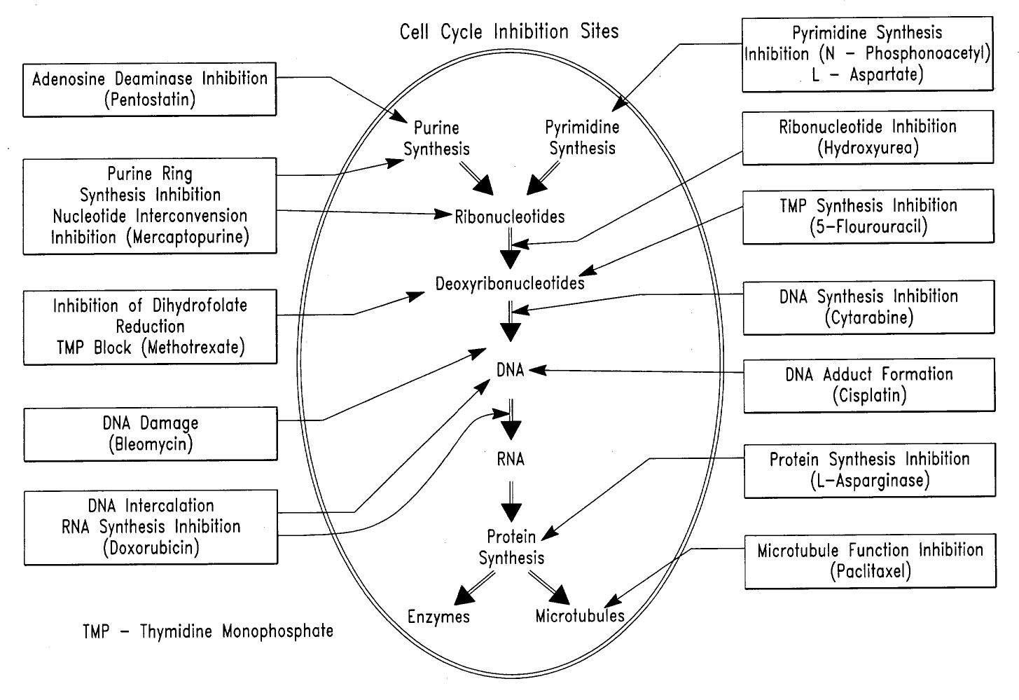

Thus, in one aspect, the Cell Cycle Inhibitor is a DNA alkylating agent. In another aspect, the Cell Cycle Inhibitor is an anti-microtubule agent. In another aspect, the Cell Cycle Inhibitor is a Topoisomerase inhibitor. In another aspect, the Cell Cycle Inhibitor is a DNA cleaving agent. In another aspect, the Cell Cycle Inhibitor is an antimetabolite. In another aspect, the Cell Cycle Inhibitor functions by inhibiting adenosine deaminase (e.g., as a purine analog). In another aspect, the Cell Cycle Inhibitor functions by inhibiting purine ring synthesis and/or as a nucleotide interconversion inhibitor (e.g., as a purine analog such as mercaptopurine). In another aspect, the Cell Cycle Inhibitor functions by inhibiting dihydrofolate reduction and/or as a thymidine monophosphate block (e.g., methotrexate). In another aspect, the Cell Cycle Inhibitor functions by causing DNA damage (e.g., Bleomycin). In another aspect, the Cell Cycle Inhibitor functions as a DNA intercalation agent and/or RNA synthesis inhibition (e.g., Doxorubicin). In another aspect, the Cell Cycle Inhibitor functions by inhibiting pyrimidine synthesis (e.g., N-phosphonoacetyl-L-Aspartate). In another aspect, the Cell Cycle Inhibitor functions by inhibiting ribonucleotides (e.g., hydroxyurea). In another aspect, the Cell Cycle Inhibitor functions by inhibiting thymidine monophosphate (e.g., 5-fluorouracil). In another aspect, the Cell Cycle Inhibitor functions by inhibiting DNA synthesis (e.g., Cytarabine). In another aspect, the Cell Cycle Inhibitor functions by causing DNA adduct formation (e.g., platinum compounds). In another aspect, the Cell Cycle Inhibitor functions by inhibiting protein synthesis (e.g., L-Asparginase). In another aspect, the Cell Cycle Inhibitor functions by inhibiting microtubule function (e.g., taxanes). In another aspect, the Cell Cycle Inhibitors acts at one or more of the steps in the biological pathway shown in FIG. 1.

Additional Cell Cycle Inhibitors useful in the present invention, as well as a discussion of their mechanisms of action, may be found in Hardman J. G., Limbird L. E. Molinoff R. B., Ruddon R W., Gilman A. G. editors, Chemotherapy of Neoplastic Diseases in Goodman and Gilman's The Pharmacological Basis of Therapeutics Ninth Edition, McGraw-Hill Health Professions Division, New York, 1996, pages 1225-1287. See also U.S. Pat. Nos. 3,387,001; 3,808,297; 3,894,000; 3,991,045; 4,012,390; 4,057,548; 4,086,417; 4,144,237; 4,150,146; 4,210,584; 4,215,062; 4,250,189; 4,258,052; 4,259,242; 4,296,105; 4,299,778; 4,367,239; 4,374,414; 4,375,432; 4,472,379; 4,588,831; 4,639,456; 4,767,855; 4,828,831; 4,841,045; 4,841,085; 4,908,356; 4,923,876; 5,030,620; 5,034,320; 5,047,528; 5,066,658; 5,166,149; 5,190,929; 5,215,738; 5,292,731; 5,380,897; 5,382,582; 5,409,915; 5,440,056; 5,446,139; 5,472,956; 5,527,905; 5,552,156; 5,594,158; 5,602,140; 5,665,768; 5,843,903; 6,080,874; 6,096,923; and RE030561 (all of which, as noted above, are incorporated by reference in their entirety)

Numerous polypeptides, proteins and peptides, as well as nucleic acids that encode such proteins, can also be used therapeutically as cell cycle inhibitors. This is accomplished by delivery by a suitable vector or gene delivery vehicle which encodes a cell cycle inhibitor (Walther & Stein, Drugs 60(2):249-71, Aug 2000; Kim et al., Archives of Pharmacal Res. 24(1): 1-15, Feb 2001; and Anwer et al., Critical Reviews in Therapeutic Drug Carrier Systems 17(4):377-424, 2000. Genes encoding proteins that modulate cell cycle include the INK4 family of genes (U.S. Pat. No. 5,889,169; U.S. Pat. No. 6,033,847), ARF-p19 (U.S. Pat. No. 5,723,313), p₂₁^{WAF1/CIP1} and p₂₇^{KIP1} (WO 9513375; WO 9835022), p27^{KIP1} (WO 9738091), p₅₇^{KIP2} (U.S. Pat. No. 6,025,480), ATM/ATR (WO 99/04266), Gadd 45 (U.S. Pat. No. 5,858,679), Myt1 (U.S. Pat. No. 5,744,349), Wee1 (WO 9949061) smad 3 and smad 4 (U.S. Pat. No. 6,100,032), 14-3-3v (WO 9931240), GSK3β (Stambolic, V. and Woodgett, J. R., Biochem Journal 303: 701-704, 1994), HDAC-1 (Furukawa, Y. et al., Cytogenet. Cell Genet. 73: 130-133, 1996; Taunton, J. et al., Science 272: 408-411, 1996), PTEN (WO 9902704), p53 (U.S. Pat. No. 5,532,220), p33^{ING1} (U.S. Pat. No. 5,986,078), Retinoblastoma (EPO 390530), and NF-1 (WO 9200387).

A wide variety of gene delivery vehicles may be utilized to deliver and express the proteins described herein, including for example, viral vectors such as retroviral vectors (e.g., U.S. Pat. Nos. 5,591,624, 5,716,832, 5,817,491, 5,856,185, 5,888,502, 6,013,517, and 6,133,029; as well as subclasses of retroviral vectors such as lentiviral vectors (e.g., PCT Publication Nos. WO 00/66759, WO 00/00600, WO 99/24465, WO 98/51810, WO 99/51754, WO 99/31251, WO 99/30742, and WO 99/15641)), alphavirus based vector systems (e.g., U.S. Pat. Nos. 5,789,245, 5,814,482, 5,843,723, and 6,015,686), adeno-associated virus-based system (e.g., U.S. Pat. Nos. 6,221,646, 6,180,613, 6,165,781, 6,156,303, 6,153,436, 6,093,570, 6,040,183, 5,989,540, 5,856,152, and 5,587,308) and adenovirus-based systems (e.g., U.S. Pat. Nos. 6,210,939, 6,210,922, 6,203,975, 6,194,191, 6,140,087, 6,113,913, 6,080,569, 6,063,622, 6,040,174, 6,033,908, 6,033,885, 6,020,191, 6,020,172, 5,994,128, and 5,994,106), herpesvirus based or “amplicon” systems (e.g., U.S. Pat. No. 5,928,913, 5,501,979, 5,830,727, 5,661,033, 4,996,152 and 5,965,441) and, “naked DNA” based systems (e.g., U.S. Pat. Nos. 5,580,859 and 5,910,488) (all of which are, as noted above, incorporated by reference in their entirety).

Within one aspect of the invention, ribozymes or antisense sequences (as well as gene therapy vehicles which can deliver such sequences) can be utilized as cell cycle inhibitors. One representative example of such inhibitors is disclosed in PCT Publication No. WO 00/32765 (which, as noted above, is incorporated by reference in its entirety).

Antiproliferative Agents.

Intimal hyperplasia is due to the migration and proliferation of cells into the intima followed by extracellular matrix secretion. The main cell types responsible for the hyperplastic response in the intima are smooth muscle cells and fibroblasts. Arterioles and capillaries sprout into the intimal plaque to provide nutrients and oxygen, thus allowing the plaque to grow. Intimal plaque growth eventually leads to occlusion of the lumen of the disease blood vessels with accompanying ischemia to the distal tissues. Hence, within one aspect of the invention, antiproliferative agents may be coated on or otherwise released from a patch.

The antiproliferative activity of the agents can be assayed by quantifying cell migration and proliferation in vitro. Antiproliferative activity can also be determined in vivo by morphometric analysis after vascular injury in various animal models (Signore et al., 2001 J. Vase. Interv. Radiol. 12: 79-88; Axel et al., 1997 Circulation 96: 636-645; Gregory et al., 1993 Transplantation 1409-1418; Burke et al., 1999 J. Cardiovasc. Pharm 33: 829-835; Poon et al., 1996 J. Clin. Invest. 2277-2283; Jones et al., 2001, J. Immunol. Methods, 254: 85-98; Gildea et al., 2000 Biotechniques 29: 81-86).

### III. Manufacture

Within certain embodiments, the compound or composition may be applied on the patch by itself or in a carrier, which may be either polymeric, or non-polymeric. Representative examples of polymeric carriers include poly (ethylene-vinyl acetate), copolymers of lactic acid and glycolic acid, poly (caprolactone), poly (lactic acid), copolymers of poly (lactic acid) and poly (caprolactone), gelatin, hyaluronic acid, collagen matrices, celluloses and albumen. Representative examples of other suitable carriers include, but are not limited to, ethanol; mixtures of ethanol and glycols (e.g., ethylene glycol or propylene glycol); mixtures of ethanol and isopropyl myristate or ethanol, isopropyl myristate and water (e.g., 55:5:40); mixtures of ethanol and eineol or D-limonene (with or without water); glycols (e.g., ethylene glycol or propylene glycol) and mixtures of glycols such as propylene glycol and water, phosphatidyl glycerol, dioleoylphosphatidyl glycerol, Transcutol®, or terpinolene; mixtures of isopropyl myristate and 1-hexyl-2-pyrrolidone, N-dodecyl-2-piperidinone or 1-hexyl-2-pyrrolidone. Other representative examples of polymer formulations are described in U.S. Pat. Nos. 5,716,981 and PCT patent application number PCT/CA00/01333, which are both incorporated by reference in their entirety.

Further examples of patents relating to polymers and their preparation include PCT Publication Nos. 98/12243, 98/19713, 98/41154, 99/07417, 00/33764, 00/21842, 00/09190, 00/09088, 00/09087, 2001/17575 and 2001/15526 (as well as their corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, and 6,214,901, which, as noted above, are all incorporated by reference in their entirety.

Patches may be coated with compositions of the present invention in a variety of manners, including for example: (a) by directly affixing to the patch a formulation (e.g., by either spraying the stent with a polymer/drug film, or by dipping the stent into a polymer/drug solution), (b) by coating the patch with a substance such as a hydrogel which will in turn absorb the composition, (c) by interweaving formulation-coated thread (or the polymer itself formed into a thread) into the patch structure, (d) by inserting the patch into a sleeve or mesh which is comprised of, or coated with, a formulation, or (e) constructing the patch itself with a composition.

Within preferred embodiments of the invention, the composition should firmly adhere to the patch during storage and at the time of implantation, and should not be dislodged from the patch when it is sutured to the blood vessel. The composition should also preferably not degrade during storage, prior to implantation, or when warmed to body temperature after implantation inside the body. In addition, it should preferably coat the patch smoothly and evenly, with a uniform distribution of agents while not changing the patch shape. Within certain preferred embodiments of the invention, the formulation should be applied to only parts of the patch, leaving the rest of the patch uncoated, for example: (a) only the luminal side of the patch is coated, (b) only the edge of the patch is coated, (c) only one end of the patch is coated, (d) a stripe is left uncoated around the patch (e) part of the patch is coated with one agent and the rest of the patch is coated with another agent.

Within one preferred embodiments of the invention, the composition should provide a predictable, prolonged release of the factor into the surrounding tissue for 1 to 12 months after implantation. Within another embodiments of the invention, the composition should provide a predictable, slow release of the factor into the surrounding tissue for 1 to 10 years after implantation. Within another embodiments of the invention, the composition should provide a predictable, prolonged release of the factor into the surrounding tissue for 1 to 4 weeks after implantation. Within another embodiments of the invention, the composition should provide a predictable, fast release of the factor into the surrounding tissue for 1 to 7 days after implantation. Within another embodiments of the invention, the composition should provide a predictable, fast release of the factor into the surrounding tissue for 1 to 24 hours after implantation. Within another embodiments of the invention, the composition is not released into the surrounding tissue. Its presence on the patch forms a chemical barrier preventing cellular adhesion to the patch, cell migration onto the patch or cell proliferation on the patch. Within certain embodiments of the invention, compositions may be combined in order to achieve a desired effect (e.g., several preparations may be combined in order to achieve both a quick and a slow or prolonged release of a given factor).

The compositions of the present invention may be formulated to contain more than one agent, to contain a variety of additional compounds, to have certain physical properties (e.g., elasticity, a particular melting point, or a specified release rate). In certain embodiments, the compositions of the instant invention are sterile.

In addition to the above properties, the composition should not cause significant turbulence in blood flow (not more than the patch itself would be expected to cause if it was uncoated).

The compositions and pharmaceutical compositions provided herein may be placed within containers, along with packaging material that provides instructions regarding the use of such materials. Generally, such instructions will include a tangible expression describing the reagent concentration, as well as within certain embodiments, relative amounts of excipient ingredients.

### IV. Application

Primary closure and patch angioplasty are two techniques of arteriotomy closure used by surgeons after vascular procedures. In primary closure, the lips of the arterial wound are directly sutured to each other whereas an extra piece of material is sutured between the two lips during patch angioplasty. Patch angioplasty is preferred after procedures with a high rate of postoperative narrowing of the repaired vessel (endarterectomy of small carotid arteries or redo operations for example). The added piece of material maintains the original diameter of the blood vessel and induces favorable local hemodynamics that otherwise may lead to recurrent stenosis (Clagett et., 1986 J Vase Surg. 3:10-23; Deriu et al., 1984 Stroke, 15: 972-979; Archie 2001 J Vase Surg. 33: 495-503; Ouriel 1987 J Vase Surg. 5:702-706; AbuRahma et al., 1998 J Vase Surg 27: 222-234; Riles et al., 1990 Surgery 107: 10-12;).

Patch angioplasty is mainly performed in two vascular procedures at the present time, carotid endarterectomy and profundaplasty. However, vascular patches are also used in other vascular procedures, for example to repair iatrogenic or traumatic arterial injuries or to repair the arterial wall after resection of a saccular aneurysm. The present invention could be applied to any vascular patching procedure.

Patch angioplasty can be performed with autologous tissue (typically a segment of the patient's veins) or synthetic material (expanded polytetrafluoroethylene or Dacron). Vein patches have drawbacks such as aneurysmal degeneration and rupture (Archie et al., Surgery 1990, 107: 389-396). They require an additional incision to harvest the vein with associated morbidity. Vein harvest also increases operative time. The patient's veins may not be suitable for patching. Most importantly, the vein used for the patch will not be available for coronary artery bypass grafting should the patient require arterial reconstruction at a later time. For these reasons, the use of synthetic patches has become increasingly popular.

Patch angioplasty improves clinical outcome in many cases but it does not afford absolute protection against recurrent carotid stenosis (Awad et al., 1989 Stroke 20: 417-422; Eikelboom et al., 1988 J Vasc Surg 7: 240-247; AbuRahma et al., 1998 J Vasc Surg 27: 222-234; AbuRahma et al., 1998 J Vasc Surg 27: 222-234; Clagett et al., 1986 J Vasc Surg 3: 10-23). Synthetic patches implanted in the vasculature induce thrombogenic, inflammatory and hyperproliferative responses. Immediately after implantation, platelets bind to the luminal surface of the prosthesis, triggering the coagulation cascade and inducing thrombus formation. Thrombus may grow large enough to cause distal ischemia. Parts of the thrombus may also become dislodged and cause embolization of distal arterioles and capillaries. In the case of carotid artery patches, thrombus occlusion and embolization lead to stroke.

In the days following the procedure, inflammatory cells such as macrophages, lymphocytes and neutrophils adhere to the prosthetic lumen and also migrate into the peri-prosthetic space. These cells release cytokines that promote smooth muscle cell migration from the adjacent vessel on the luminal surface of the patch. The cells further proliferate on the patch and secrete extracellular matrix. Depending on the porosity of the patch material, cells may also migrate through the pores of the patch from the surrounding tissue into the lumen. In both cases, hyperplasia causes plaque formation on the luminal surface of the patch and the adjacent vessels within a few weeks. This reduces luminal area in the treated blood vessel, thus impeding blood flow to the distal tissue. The present invention involves coating synthetic patches with agents preventing inflammatory reaction, thrombus formation and intimal hyperplasia in order to inhibit restenosis of the treated vessel.

A. Carotid Endarterectomy

A 10-cm long skin incision is made along the anterior border of the sternocleidomastoid muscle. After retraction of the muscle, the distal common carotid artery, the carotid bifurcation and the proximal segments of the internal and external carotid arteries are dissected. The three vessels are clamped. An arteriotomy is made in the common carotid artery extending antero-laterally through the plaque into the internal carotid artery beyond the distal extension of the plaque. The intimo-medial layer of the plaque is transected in the common carotid and the plaque is excised to the adventitia. A coated patch is trimmed and tapered to appropriate size (typically 7 cm long with a 4 mm apex and a 7 mm bulb). The coated patch is placed along the edges of the arteriotomy to reconstruct the original shape of the vessel and to replace a significant portion of the endarterectomized wall of the artery. The coated patch is sutured to the edges of the arteriotomy with a continuous 7-0 polypropylene suture. Blood flow is restored by releasing all clamps and the skin wound is closed.

B. Profundaplasty

The common femoral artery and the profunda femoris artery (PFA) are isolated through a vertical groin incision. Once the branches distal to the end of the occlusive disease are controlled, the common femoral, superficial femoral and the PFA branches are clamped. An arteriotomy is performed, starting on the common femoral and extending down the PFA until the plaque ends. Endarterectomy of the involved common femoral and PFA is performed as needed. A coated patch is trimmed to size to achieve a smooth taper in the PFA to re-establish optimal flow characteristics in the repaired vessel. The coated patch is sutured to the edges of the arteriotomy with a continuous 7-0 polypropylene suture. Blood flow is restored by releasing all clamps and the skin wound is closed.

It should be obvious to one of skill in the art that the above-described compositions can be utilized to create variation in the Examples provided below, without deviating from the spirit and scope of the invention.

### EXAMPLES

### Example 1

### Manufacture of Coated Patches

A. Procedure for Sprayed Patches

The following describes a typical method using an oval 2 cm×0.5 cm synthetic patch. For larger patches, larger volumes of polymer/drug solution are used.

Briefly, a sufficient quantity of polymer is weighed directly into a 20 mL glass scintillation vial, and sufficient DCM added in order to achieve a 2% w/v solution. The vial is then capped and mixed by hand in order to dissolve the polymer. The patch is then held in a vertical orientation with micro clamps connected to a holding apparatus 6 to 12 inches above the fume hood floor to enable horizontal spraying. Using an automatic pipette, a suitable volume (minimum 5 ml) of the 2% polymer solution is transferred to a separate 20 ml glass scintillation vial. An appropriate amount of paclitaxel is then added to the solution and dissolved by hand shaking.

To prepare for spraying, remove the cap of this vial and dip the barrel of a TLC atomizer into the polymer solution. Note that the reservoir of the atomizer need not be used in this procedure: the 20 ml glass vial acts as a reservoir. Connect the nitrogen tank to the gas inlet of the atomizer. Gradually increase the pressure until atomization and spraying begins. Note the pressure and use this pressure throughout the procedure. To spray the patch use 5 second oscillating sprays with a 15 second dry time between sprays. After 5 sprays, rotate the patch 180° and spray the other side of the patch. During the dry time, finger crimp the gas line to avoid wastage of the spray. Spraying is continued until a suitable amount of polymer is deposited on the patch. The amount may be based on the specific patching application in vivo. To determine the amount, weigh the patch after spraying has been completed and the patch has dried. Subtract the original weight of the patch from the finished weight. This produces the amount of polymer (plus paclitaxel) applied to the patch. Store the coated patch in a sealed container.

B. Procedure for Dipped Patches

The following describes a typical method using a 2 cm×0.5 cm oval synthetic patch. For larger patches, larger volumes of polymer/drug solution are used.

Weigh 2 g of polymer into a 20 mL glass scintillation vial and add 20 mL of DCM. Cap the vial and leave it for 2 hours to dissolve (hand shake the vial frequently to assist the dissolving process). Weigh a known amount of paclitaxel directly into an 8 mL glass vial and add 4 mL of the polymeric solution. Using a glass Pasteur pipette, dissolve paclitaxel by gently pumping the polymer solution. Once paclitaxel is dissolved, hold the glass vial in a near horizontal position (the sticky polymer solution will not flow out). Using tweezers, insert the patch into the vial all the way to the bottom. Allow the polymer solution to flow almost to the mouth of the vial by angling the mouth below horizontal and then restoring the vial to an angle slightly above the horizontal. Slowly remove the patch (approximately 30 seconds). Hold the patch in a vertical position to dry.

### Example 2

### In Vitro Drug Release Rate

Small pieces (0.5×0.5 cm) of paclitaxel-coated patches (n=4) are placed in 14 mL glass tubes followed by 10 mL phosphate buffered saline (PBS, pH=7.4) containing 0.4 g/L albumin. The tubes are incubated at 37° C. with gentle rotational mixing at 8 rpm. At regular time intervals, 10 mL of supernatant are withdrawn for paclitaxel analysis and replaced with fresh PBS/albumin buffer. One mL of dichloromethane is added to the withdrawn supernatant and the tube is capped and shaken by hand for 1 minute to allow all the released paclitaxel to partition into the separate dichloromethane phase. The tubes are then centrifuged at 500×g for 1 minute, the 10 mL of top aqueous phase are withdrawn and discarded and the dichloromethane phase is evaporated under nitrogen at 50° C. for 20 minutes. One mL of a 60% acetonitrite in water (v/v) solution is added to each tube to solubilize the dried contents. These solutions are then analyzed for paclitaxel by HPLC using a Waters C18 Novapak column with a mobile phase composed of 58% acetonitrite/5% methanol/37% water at a flow rate of 1 mL/minute with detection at 232 nm. The HPLC method for quantitation of the released drug is chosen over other methods, such as radiolabelled assays, because the chromatographic method ensures that only paclitaxel molecules in the intact (non-degraded) form are measured. A standard curve of paclitaxel dissolved in 60% acetonitrile: 40% water is obtained in the 0-50 μg/mL range and used to directly quantitate the amount of paclitaxel released.

### Example 3

### In Vivo Patch Efficacy

General anesthesia is induced into domestic swine. The neck region is shaved and the skin sterilized with cleansing solution. A vertical incision is made under sterile condition on one side of the neck and the common carotid artery is exposed. Two vascular clamps are placed on the artery to temporarily stop blood flow and an arteriotomy is performed between the clamps. The arteriotomy is closed with a synthetic patch. The animals are randomized into 4 groups of 5 pigs receiving a synthetic patch coated with (1) carrier polymer alone, (2) carrier polymer loaded with 1% paclitaxel, (3) carrier polymer loaded with 5% paclitaxel or (4) carrier polymer loaded with 10% paclitaxel. The clamps are released and the skin is closed.

The contralateral carotid artery is prepared in the same manner and a control uncoated patch is used to repair the arteriotomy. The animal is recovered.

The animals are sacrificed at 1 month and perfused with saline followed by 10% phosphate buffered formaldehyde for 30 minutes under 100 mmHg pressure. The carotid arteries are removed and kept in the same fixative solution overnight. The specimens are then prepared for histology. Cross sections are cut and stained with H&E and Movat's stains. Histopathology of the tissue surrounding the patch is recorded. Morphometric analysis is performed to measure hyperplasia on the luminal surface of the patch and in adjacent vessels.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

I claim:

1. A surgical patch which releases at least one of an anti-inflammatory agent, an anti-platelet agent, an anticoagulant agent, a fibrinolytic agent, a cell-cycle inhibitor agent, and an anti-proliferative agent.

2. The surgical patch according to claim 1 wherein said patch is coated with one or more of said agents.

3. The surgical patch according to claim 1 wherein said agent further comprises a polymer.

4. The surgical patch according to claim 3 wherein said patch is a vascular patch.

5. The surgical patch according to claim 3 wherein said patch releases an anti-inflammatory agent.

6. The surgical patch according to claim 3 wherein said anti-inflammatory agent is aspirin, ibuprofen, or a glucocorticoid drug.

7. The surgical patch according to claim 3 wherein said anti-coagulant agent is heparin or hirudin.

8. The surgical patch according to claim 3 wherein said fibrinolytic agent is tissue plasminogen activator, streptokinase, or urokinase.

9. The surgical patch according to any one of claims 1 or 3 wherein said cell cycle inhibitor is a taxane, a vinca alkaloid, a camptothecin, a podophyllotoxin, an anthracycline, a platinum compound, a nitrosourea, a nitroiidazole, a folic acid antagonist, a cytidine analog, a pyrimidine analog, a purine analog, a nitrogen mustard, a hydroxyurea, a mytomycin, a benzamide, or a tetrazine.

10. The surgical patch according to claim 9 wherein said taxane is paclitaxel.

11. The surgical patch according to claim 9 wherein said vinca alkaloid is vinblastine or vincristine.

12. The surgical patch according to claim 9 wherein said podophyllotoxin is etoposide.

13. The surgical patch according to claim 9 wherein said anthracycline is doxorubicin or mitoxantrone.

14. The surgical patch according to claim 9 wherein said platinum compound is cisplatin or carboplatin.

15. The surgical patch according to claim 1 wherein said patch releases at least two or more of said agents.

16. The surgical patch according to claim 15 wherein said patch releases both an anti-inflammatory agent and a cell-cycle inhibitor agent.

17. The surgical patch according to claim 1 wherein said patch is comprised of a synthetic material.

18. The surgical patch according to claim 1 wherein said patch is comprised of a biological tissue.

19. A method for closing an opening in a biological tissue, comprising applying a surgical patch according to any one of claims 1, 3, 17, or 18 to said opening.

20. The method according to claim 19 wherein said surgical patch is sutured in place.

21. The method according to claim 19 wherein said surgical patch is a vascular patch.

22. A method for making a drug-loaded surgical patch, comprising coating all or a portion of a surgical patch with at least one of an anti-inflammatory agent, an anti-platelet agent, an anticoagulant agent, a fibrinolytic agent, a cell-cycle inhibitor agent, and an anti-proliferative agent.

23. The method according to claim 22 wherein said patch is coated by dipping or spraying said agent on said patch.

24. The method according to claim 22 wherein said patch is coated with two or more of said agents.

25. The method according to claim 22 wherein said agent further comprises a polymer.

26. The method according to claim 22 wherein said patch is a vascular patch.

27. The method according to claim 25 wherein said patch releases an anti-inflammatory agent.

28. The method according to claim 25 wherein said anti-inflammatory agent is aspirin, ibuprofen, or a glucocorticoid drug.

29. The method according to claim 25 wherein said anti-coagulant agent is heparin or hirudin.

30. The method according to claim 25 wherein said fibrinolytic agent is tissue plasminogen activator, streptokinase, or urokinase.

31. The method according to any one of claims 22 or 25 wherein said cell cycle inhibitor is a taxane, a vinca alkaloid, a camptothecin, a podophyllotoxin, an anthracycline, a platinum compound, a nitrosourea, a nitroiidazole, a folic acid antagonist, a cytidine analog, a pyrimidine analog, a purine analog, a nitrogen mustard., a hydroxyurea, a mytomycin, a benzamide, or a tetrazine.

32. The method according to claim 31 wherein said taxane is paclitaxel.

33. The method according to claim 31 wherein said vinca alkaloid is vinblastine or vincristine.

34. The method according to claim 31 wherein said podophyllotoxin is etoposide.

35. The method according to claim 31 wherein said anthracycline is doxorubicin or mitoxantrone.

36. The method according to claim 31 wherein said platinum compound is cisplatin or carboplatin.

37. The method according to claim 22 wherein said patch releases at least two or more of said agents when applied to an opening in a biological tissue.

38. The method according to claim 37 wherein said patch releases both an anti-inflammatory agent and a cell-cycle inhibitor agent.

39. The method according to claim 22 wherein said patch is comprised of a synthetic material.

40. The method according to claim 22 wherein said patch is comprised of a biological tissue.
